(19) United States
(12) Patent Application Publication
(10) Pub. No.: US 2025/0312264 A1
(43) Pub. Date: Oct. 9, 2025

(54) Skin Care Compositions

(71) Applicant: Colgate Palmolive Company, New York, NY (US)

(72) Inventors: Marwa EL HAJOUI, Carrières Sous Poissy (FR); Benedicte Deschamps, Paris (FR); Nadege Durand, Cursan (FR); Jin Namkoong, High Bridge, NJ (US); Roxane Henry, Poissy (FR); Colline Haxaire, Ridgewood, NJ (US); Alexandra D'Arcangelis, Randolph, NJ (US); Sayantani Goswami Chatterjee, Edison, NJ (US)

(73) Assignee: Colgate-Palmolive Company, New York, NY (US)

(21) Appl. No.: 19/172,145

(22) Filed: Apr. 7, 2025

(30) Foreign Application Priority Data
Apr. 5, 2024 (FR) FR2403549
Apr. 5, 2024 (FR) FR2403557

(51) Int. Cl. A61K 8/73; A61K 8/44; A61K 8/64; A61K 8/67; A61K 8/9789; A61Q 19/08

(52) U.S. Cl. CPC A61K 8/735; A61K 8/64; A61K 8/673; A61K 8/678; A61K 8/9789; A61Q 19/08; A61K 8/44; A61K 2800/5922; A61K 2800/91

(57) ABSTRACT

Skin care compositions and, preferably, skin care compositions providing one or more improvements in skin appearance and, in some cases, also in skin health are disclosed herein. In accordance with an aspect of the invention, provided is a skin care composition comprising hyaluronic acid or a salt thereof; vitamin E; a peptide; an extract comprising one or more of silybum marianum extract or pseudoalteromonas ferment extract; and water in an amount of about 45 wt. % or more, based on the total weight of the skin care composition.

### CROSS-REFERENCE TO RELATED APPLICATIONS

This application claims the benefit of priority from French Patent Application No. FR2403557, filed Apr. 5, 2024, titled Skin Care Compositions, and from French Patent Application No. FR2403549, filed Apr. 5, 2024, titled Skin Care Compositions, both of which are incorporated herein by reference in their entireties for all purposes.

### BACKGROUND

Reactive oxygen species (ROS) are free radicals that can react with various cellular proteins in the skin, for example, collagen, elastin and glycosaminoglycans. ROS causes cross-linking of collagen and elastin resulting in wrinkles and decreasing the skin's ability to repair itself. Thus, the skin damage caused by ROS can contribute to the physiology of aging or lead to skin disorders. Antioxidants can scavenge and decrease the excessive ROS produced and mitigate its damage to the cells of different tissues, including skin.

Furthermore, in the field of skin health, recent consumer trends are requiring products be as simple as possible but still provide acceptable skin health benefits that consumers have grown accustomed to in products with a number of ingredients. However, when using fewer ingredients, there is more of an emphasis on maximizing the results and efficacy of each ingredient in order to delivery anti-oxidant and skin health benefits to a consumer.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to skin care compositions and, preferably, skin care compositions providing one or more improvements in skin appearance and, in some cases, also in skin health. In accordance with an aspect of the invention, provided is a skin care composition comprising hyaluronic acid or a salt thereof; vitamin E; a peptide; an extract comprising one or more of silybum marianum extract or pseudoalteromonas ferment extract; and water in an amount of about 45 wt. % or more, based on the total weight of the skin care composition.

In accordance with another aspect of the invention, provided is a method for reducing wrinkles and/or improving skin elasticity, homogeneity, luminosity, and/or firmness. In some embodiments, the method comprises applying a skin care composition to skin, wherein the skin care composition comprises hyaluronic acid or a salt thereof, vitamin E, a peptide, an extract comprising one or more of silybum marianum extract or pseudoalteromonas ferment extract, and water in an amount of about 45 wt. % or more, based on the total weight of the skin care composition.

In accordance with further aspect of the invention, provided is a method comprising: injecting a composition to an area of skin; and applying a serum to the area of skin, the serum comprising: hyaluronic acid or a salt thereof, vitamin E, a peptide, an extract comprising one or more of silybum marianum extract or pseudoalteromonas ferment extract, and water in an amount of about 45 wt. % or more, based on the total weight of the skin care composition.

A list of non-limiting example embodiments in accordance with aspects of the invention is provided:

In accordance with an embodiment 1, provided is a skin care composition comprising: hyaluronic acid or a salt thereof; vitamin E; a peptide; an extract comprising one or more of silybum marianum extract or pseudoalteromonas ferment extract; and water in an amount of about 45 wt. % or more, based on the total weight of the skin care composition.

In accordance with an embodiment 2, provided is the skin care composition according to embodiment 1, wherein the hyaluronic acid or a salt thereof comprises hydrolyzed hyaluronic acid.

In accordance with an embodiment 3, provided is the skin care composition according to embodiment 1 or embodiment 2, wherein the hyaluronic acid or a salt thereof comprises sodium hyaluronate.

In accordance with an embodiment 4, provided is the skin care composition according to any foregoing embodiment, wherein the vitamin E is selected from alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, and a combination of two or more thereof.

In accordance with an embodiment 5, provided is the skin care composition according to any foregoing embodiment, wherein the vitamin E comprises alpha-tocopherol.

In accordance with an embodiment 6, provided is the skin care composition according to any foregoing embodiment, wherein the peptide comprises dipeptide diaminobutyroyl benzylamide diacetate, acetyl tetrapeptide-11, acetyl tetrapeptide-9, palmitoyl tripeptide-1, palmitoyl tetrapeptide-7, or a combination of two or more thereof.

In accordance with an embodiment 7, provided is the skin care composition according to any foregoing embodiment, wherein the peptide is selected from dipeptides.

In accordance with an embodiment 8, provided is the skin care composition according to any foregoing embodiment, wherein the peptide comprises dipeptide diaminobutyroyl benzylamide diacetate.

In accordance with an embodiment 9, provided is the skin care composition according to any foregoing embodiment, wherein the extract comprises both silybum marianum extract and pseudoalteromonas ferment extract.

In accordance with an embodiment 10, provided is the skin care composition according to any foregoing embodiment further comprising one or more polyol, optionally, in an amount from about 4 to about 22 wt. %, based on the total weight of the skin care composition.

In accordance with an embodiment 11, provided is the skin care composition according to embodiment 10, wherein the one or more polyol comprises a plurality of polyols, the plurality of polyols comprising glycerin and one or more glycol.

In accordance with an embodiment 12, provided is the skin care composition according to embodiment 10 or embodiment 11, wherein the one or more polyol comprises ethylene glycol, propylene glycol, butylene glycol, caprylyl glycol hexylene glycol, diethylene glycol, dipropylene glycol, or a combination of two or more thereof.

In accordance with an embodiment 13, provided is the skin care composition according to any foregoing embodiment further comprising: a nonionic surfactant, optionally, in an amount from about 0.1 to about 5 wt. %, based on the total weight of the skin care composition.

In accordance with an embodiment 14, provided is the skin care composition according to embodiment 13, wherein the nonionic surfactant is selected from pegylated castor oils, polyglycerol esters, poloxamers, fatty alcohol ethoxylates, alkylphenol ethoxylates, tertiary amine oxides, glucosides, and a combination of two or more thereof.

In accordance with an embodiment 15, provided is the skin care composition according to embodiment 14, wherein the pegylated castor oil has a pegylated chain of about 10 to about 80.

In accordance with an embodiment 16, provided is the skin care composition according to any foregoing embodiment further comprising: an amino acid component comprising ergothioneine.

In accordance with an embodiment 17, provided is the skin care composition according to embodiment 16, wherein the amino acid component comprises taurine, arginine, glycine, or a combination of two or more thereof.

In accordance with an embodiment 18, provided is the skin care composition according to embodiment 17, wherein the amino acid component comprises taurine, arginine, and glycine.

In accordance with an embodiment 19, provided is the skin care composition according to any foregoing embodiment further comprising: vitamin B, optionally, present in an amount from about 0.1 to about 3 wt. %, based on the total weight of the skin care composition.

In accordance with an embodiment 20, provided is the skin care composition according to embodiment 19, wherein the vitamin B comprises niacinamide.

In accordance with an embodiment 21, provided is the skin care composition according to any foregoing embodiment further comprising: a pyrrolidone carboxylic acid and/or a salt thereof, optionally, present in an amount from about 0.1 to about 3 wt. %, based on the total weight of the skin care composition.

In accordance with an embodiment 22, provided is the skin care composition according to embodiment 21, wherein the skin care composition has a weight ratio of pyrrolidone carboxylic acid and/or a salt thereof to niacinamide of from about 1:1 to about 1:11.

In accordance with an embodiment 23, provided is the skin care composition according to one of embodiments 16 to 22, wherein the skin care composition has a weight ratio of alpha-tocopherol to ergothioneine of about 6:1 to about 1:6.

In accordance with an embodiment 24, provided is the skin care composition according to one of embodiments 20 to 23, wherein the skin care composition has a weight ratio of alpha-tocopherol to niacinamide of from about 1:1 to about 1:11.

In accordance with an embodiment 25, provided is the skin care composition according to one of embodiments 20 to 24, wherein the skin care composition has a weight ratio of ergothioneine to niacinamide of from about 1:1 to about 1:11.

In accordance with an embodiment 26, provided is the skin care composition according to any foregoing embodiment, wherein the water is present in an amount from about 60 to about 90wt. %, optionally about 70 to about 90 wt. % or about 75 to about 80 wt. %, based on the total weight of the skin care composition.

In accordance with an embodiment 27, provided is a method of reducing wrinkles and/or improving skin elasticity, homogeneity, luminosity, and/or firmness, the method comprising: injecting a composition to an area of skin; and applying a serum to the area of skin, the serum comprising: hyaluronic acid or a salt thereof, vitamin E, a peptide, an extract comprising one or more of silybum marianum extract or pseudoalteromonas ferment extract, and water in an amount of about 45 wt. % or more, based on the total weight of the skin care composition.

In accordance with an embodiment 28, provided is the skin care composition according to embodiment 27, wherein the serum is applied to the area of skin within 72 hours of the injection of the composition.

It should be understood that the detailed description and specific examples, while indicating some typical aspects of the disclosure, are intended for purposes of illustration only and are not intended to limit the scope of the disclosure.

### BRIEF DESCRIPTION OF THE DRAWINGS

Implementation of the present technology will now be described, by way of example only, with reference to the attached figures, wherein:

FIG. 1 is a bar graph showing the reduction of oxidative species after application of non- limiting exemplary compositions in accordance with an aspect of the invention;

FIG. 2 is a bar graph of the effect on elastin fibers after application of non-limiting exemplary compositions in accordance with an aspect of the invention;

FIG. 3 is a bar graph of the effect on COL1A1, FBN1, COL7A1, LUM, and TNC expression after application of non-limiting exemplary compositions in accordance with an aspect of the invention;

FIG. 4 is a bar graph of the effect on loricrin expression after application of non-limiting exemplary compositions in accordance with an aspect of the invention;

FIG. 5 is a bar graph of the effect on CASPASE 9 gene expression after application of a non-limiting exemplary composition or comparative compositions in accordance with an aspect of the invention; and

FIG. 6 is a bar graph of the effect on H2AFJ gene expression after application of a non-limiting exemplary composition or comparative compositions in accordance with an aspect of the invention.

It should be understood that the various aspects are not limited to the arrangements and instrumentality shown in the drawings.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the skin care composition. The symbol “0” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the skin care compositions of the instant disclosure can be free or essentially free of all components and elements positively recited throughout the instant disclosure. In some instances, the skin care compositions of the present disclosure may be substantially free of non-incidental amounts of the ingredient(s) or compound(s) described herein. A non-incidental amount of an ingredient or compound is the amount of that ingredient or compound that is added into the skin care composition by itself. For example, a skin care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the skin care composition includes both components (or the composition includes more than two components that overlap), an overlapping compound does not represent more than one component. For example, certain compounds may be characterized as both a surfactant and an emulsifier. If a particular skin care composition includes both a surfactant and an emulsifier, a compound characterized as both a surfactant and an emulsifier will serve only as either a polyol or a sweetener—not both.

In some instances, the skin care compositions of the present disclosure may be substantially free of non-incidental amounts or added amounts of the ingredient(s) or compound(s) described herein. A non-incidental amount of an ingredient or compound is the amount of that ingredient or compound that is added into the skin care composition by itself. For example, a skin care composition may be substantially free of a non-incidental amount of an ingredient or compound, although such ingredient(s) or compound(s) may be present as part of a raw material that is included as a blend of two or more compounds.

For readability purposes, the chemical functional groups are in their adjective form; for each of the adjectives, the word “group” is assumed. For example, the adjective “alkyl” without a noun thereafter, should be read as “an alkyl group.”

As used herein, the term “subject” refers to individuals (e.g., human) to be treated by the methods or compositions of the present invention. Subjects include, but are not limited to, mammals (e.g., murines, simians, equines, bovines, porcines, canines, felines, and the like), and most preferably includes humans. In the context of the invention, the term “subject” generally refers to an individual who will receive or who has received treatment for a condition characterized by the presence of biofilm-forming bacteria, or in anticipation of possible exposure to biofilm-forming bacteria.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

Aspects of the invention are directed to skin care compositions and, preferably, skin care compositions providing one or more improvements in skin appearance and, in some cases, also in skin health. In accordance with an aspect of the invention, provided is a skin care composition comprising hyaluronic acid or a salt thereof; vitamin E; a peptide; an extract comprising one or more of silybum marianum extract or pseudoalteromonas ferment extract; and water in an amount of about 45 wt. % or more, based on the total weight of the skin care composition.

In accordance with another aspect of the invention, provided is a method for reducing wrinkles and/or improving skin elasticity, homogeneity, luminosity, and/or firmness. In some embodiments, the method comprises applying a skin care composition to skin, wherein the skin care composition comprises hyaluronic acid or a salt thereof, vitamin E, a peptide, an extract comprising one or more of silybum marianum extract or pseudoalteromonas ferment extract, and water in an amount of about 45 wt. % or more, based on the total weight of the skin care composition.

In accordance with further aspect of the invention, provided is a method comprising: injecting a composition to an area of skin; and applying a serum to the area of skin, the serum comprising: hyaluronic acid or a salt thereof, vitamin E, a peptide, an extract comprising one or more of silybum marianum extract or pseudoalteromonas ferment extract, and water in an amount of about 45 wt. % or more, based on the total weight of the skin care composition. The serum may be applied to the area of skin within 72 hours of the injection of the composition in some instances.

In accordance with yet another aspect of the invention, provided is a skin care composition comprising a hyaluronic acid or a salt thereof; vitamin E; a peptide; an extract comprising silybum marianum extract; optionally a pseudoalteromonas ferment extract; and water in an amount of about 45 wt. % or more, based on the total weight of the skin care composition.

The skin care composition and/or a method thereof may preferably reduce the red spots count on an individual's skin presenting red spots by about 2.8% or more after one day of applying twice a day (e.g., once in the morning and once in the evening) and/or by about 10.1% or more after 7 days of applying twice a day (e.g., once in the morning and once in the evening) to the individual's skin presenting the red spots. Additionally or alternatively, the skin care composition and/or a method thereof may preferably reduce the red spots' area on an individual's skin presenting red spots by about 7.9% or more after one day of applying twice a day (e.g., once in the morning and once in the evening), by about 18.8% or more after 7 days of applying twice a day (e.g., once in the morning and once in the evening), and/or by about 20.1% or more after 28 days of applying twice a day (e.g., once in the morning and once in the evening) to the individual's skin presenting the red spots.

The skin care composition and/or a method thereof may preferably improve the erythema assessment score on an individual's skin presenting erythema by about 3.9% or more after one day of applying twice a day (e.g., once in the morning and once in the evening), by about 4.4% or more after 7 days of applying twice a day (e.g., once in the morning and once in the evening), and/or by about 22.8% or more after 28 days of applying twice a day (e.g., once in the morning and once in the evening) to the individual's skin presenting the erythema.

The skin care compositions may be in the form of a serum, a lotion, a cream, a balm, a gel, a mesotherapy injection composition, and/or the like. In certain preferred embodiments, the skin care composition is a topical skin care composition, which may be in the form of a serum, a lotion, a cream, a balm, and/or the like. In some embodiments, the skin care composition is in the form selected from: a serum, a cream, a moisturizer, a mask, a cleanser, a facial make-up, and a balm (e.g., lip balm). In some aspects, skin care compositions of the disclosure can be applied by injection (e.g., subcutaneous or dermal injection). In certain aspects, skin care composition is intended to refer to a product for use at home or in a professional setting. The skin care composition may be in the form of an injectable mesotherapy composition in some instances.

In some embodiments, the skin care compositions may have a single phase, which contains all of the components and/or ingredients of the skin care composition. In other embodiments, the skin care composition may include two or more phases, such as two, three, four, or five phases. The composition can comprise a single phase or can be a multi-phase system, for example a system comprising a polar phase and an oil phase, optionally in the form of a stable emulsion. For example, the skin care composition may be in the form of an emulsion having more than one phase, such as two phases or three phases. In some cases, the skin care composition may be in the form of an oil in water emulsion or a water in oil emulsion.

Suitable components, such as those listed below, may be included or excluded from the formulations for the skin care compositions depending on the specific combination of other ingredients and the form of the skin care compositions. The skin care compositions of the disclosure can be liquid, semi-solid or solid. The skin care compositions can be provided in any suitable container, tube, or container with a porous cap, roll-on container, bottle, container with an open end, etc.

The skin care compositions typically comprise hyaluronic acid and/or a salt thereof. The hyaluronic acid and/or a salt thereof may comprise hyaluronic acid in the form of a salt, such as sodium hyaluronate. In some embodiments, the hyaluronic acid and/or a salt thereof consists of hyaluronic acid or consists of non-incidental amounts of hyaluronic acid. In other embodiments, the hyaluronic acid and/or a salt thereof consists of salts of hyaluronic acid (e.g., sodium hyaluronate) or non-incidental amounts of salts of hyaluronic acid.

The amount of hyaluronic acid and/or a salt thereof in the skin care composition may be from about 0.05 to about 5 wt. %, based on the total weight of the skin care composition. For example, the total amount of hyaluronic acid and/or a salt thereof in the skin care composition may be from about 0.05 to about 5 wt. %, about 0.05 to about 4 wt. %, about 0.05 to about 3 wt. %, about 0.05 to about 2 wt. %, about 0.05 to about 1.5 wt. %, about 0.05 to about 1 wt. %, about 0.05 to about 0.8 wt. %, about 0.05 to about 0.6 wt. %, about 0.05 to about 0.4 wt. %; from about 0.1 to about 5 wt. %, about 0.1 to about 4 wt. %, about 0.1 to about 3 wt. %, about 0.1 to about 2 wt. %, about 0.1 to about 1.5 wt. %, about 0.1 to about 1 wt. %, about 0.1 to about 0.8 wt. %, about 0.1 to about 0.6 wt. %, about 0.1 to about 0.4 wt. %; from about 0.2 to about 5 wt. %, about 0.2 to about 4 wt. %, about 0.2 to about 3 wt. %, about 0.2 to about 2 wt. %, about 0.2 to about 1.5 wt. %, about 0.2 to about 1 wt. %, about 0.2 to about 0.8 wt. %, about 0.2 to about 0.6 wt. %, about 0.2 to about 0.4 wt. %; from about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1 wt. %; from about 0.75 to about 5 wt. %, about 0.75 to about 4 wt. %, about 0.75 to about 3 wt. %, about 0.75 to about 2 wt. %, about 0.75 to about 1.5 wt. %; from about 1 to about 5 wt. %, about 1 to about 4 wt. %, about 1 to about 3 wt. %, about 1 to about 2 wt. %, about 1 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition. Without being limited to any particular theory, certain embodiments of the skin care composition can achieve desirable improvements in the appearance and/or health of the skin while employing a total amount of hyaluronic acid and/or a salt thereof of about 1 wt. % or less, about 0.8 wt. % or less, about 0.6 wt. % or less, about 0.5 wt. % or less, about 0.4 wt. % or less, or any range formed from the foregoing ranges and upper limits disclosed in this paragraph, wherein the weight percentage is based on the total weight of the skin care composition.

The hyaluronic acid and/or a salt thereof may have a low molecular weight (e.g., an average molecular weight of 300 kDA or less). In some embodiments, the hyaluronic acid and/or a salt thereof has an average molecular weight (MW) of about 100,000 Da or less. For example, the molecular weight of the hyaluronic acid and/or a salt thereof is from about 10 kDa to about 75 kDa, from about 25 kDa to 75 kDa, about 35 kDa to about 75 kDa, about 40 kDa to about 75 kDa, about 45 kDa to about 75 kDa, about 50 kDa to about 75 kDa, about 55 kDa to about 75 kDa, about 60 kDa to about 75 kDa; from about 10 kDa to about 65 kDa, about 25 kDa to 65 kDa, about 35 kDa to about 65 kDa, about 40 kDa to about 65 kDa, about 45 kDa to about 65 kDa, about 50 kDa to about 65 kDa, about 55 kDa to about 65 kDa; from about 10 kDa to about 60 kDa, about 25 kDa to 60 kDa, about 35 kDa to about 60 kDa, about 40 kDa to about 60 kDa, about 45 kDa to about 60 kDa, about 50 kDa to about 60 kDa; from about 10 kDa to about 55 kDa, about 25 kDa to 55 kDa, about 35 kDa to about 55 kDa, about 40 kDa to about 55 kDa, about 45 kDa to about 55 kDa; from about 10 kDa to about 50 kDa, about 25 kDa to 50 kDa, about 35 kDa to about 50kDa, about 40 kDa to about 50 kDa; from about 10 kDa to about 45 kDa, about 25 kDa to 45 kDa; from about 10 kDa to about 40 kDa, about 25 kDa to 40 kDa, about 30 kDa to about 40 kDa, or any range or subrange thereof. In at least one embodiment, the skin care composition comprises hyaluronic acid having a molecular weight of about 50 kDa.

The skin care composition also typically comprises a vitamin E. As used herein, unless otherwise specified, “vitamin E” refers to a family of four isomers of tocopherols and four isomers of tocotrienols. All eight isomers of vitamin E have a 6-chromanol ring structure and a side chain. The four tocopherols include fully saturated side chains and include alpha-tocopherol, gamma-tocopherol, beta-tocopherol, and delta-tocopherol. The four tocotrienols include unsaturated side chains and include alpha-tocotrienol, gamma-tocotrienol, beta-tocotrienol, and delta-tocotrienol. For example, vitamin E may be or include one or more of alpha-tocopherol, gamma-tocopherol, beta-tocopherol, delta-tocopherol, alpha-tocotrienol, gamma-tocotrienol, beta-tocotrienol, delta-tocotrienol, or any combination thereof. In at least one implementation, the vitamin E includes at least one of the four tocopherols. It should be appreciated that the vitamin E and/or the isomers thereof may be or include natural forms of vitamin E, synthetic forms of vitamin E, or combinations thereof. Any one or more of the isomers of vitamin E may be in the “d” form, the “l” form, or mixtures thereof. In some embodiments, vitamin E is vitamin E acetate or Vitamin E succinate. In some embodiments, vitamin E is tocopheryl acetate.

In some embodiments, the skin care composition includes one or more vitamin E selected from vitamin E acetate, vitamin E succinate, vitamin E acetate, or a combination of two or more thereof. The vitamin E may, in some cases, be selected from the group consisting of alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, natural extracts enriched in vitamin E derivatives, and combinations of two or more thereof. In at least one instance, the vitamin E comprises tocopherol (e.g., alpha-tocopherol) or topically acceptable salts thereof.

The total amount of vitamin E in the skin care compositions may be from about 0.05 to about 5 wt. %, based on the total weight of the skin care composition. For example, the total amount of vitamin E in the skin care composition may be from about 0.05 to about 3 wt. %, about 0.05 to about 1.5 wt. %, about 0.05 to about 1 wt. %, about 0.05 to about 0.8 wt. %, about 0.05 to about 0.6 wt. %, about 0.05 to about 0.4 wt. %; from about 0.1 to about 5 wt. %, about 0.1 to about 3 wt. %, about 0.1 to about 1.5 wt. %, about 0.1 to about 1 wt. %, about 0.1 to about 0.8 wt. %, about 0.1 to about 0.6 wt. %, about 0.1 to about 0.4 wt. %; from about 0.2 to about 5 wt. %, about 0.2 to about 3 wt. %, about 0.2 to about 1.5 wt. %, about 0.2 to about 1 wt. %, about 0.2 to about 0.8 wt. %, about 0.2 to about 0.6 wt. %, about 0.2 to about 0.4 wt. %; from about 0.5 to about 5 wt. %,, about 0.5 to about 3 wt. %, about 0.5 to about 1.5 wt. %, about 0.5 to about 1 wt. %; from about 0.75 to about 5 wt. %, about 0.75 to about 3 wt. %, about 0.75 to about 1.5 wt. %; from about 1 to about 5 wt. %, about 1 to about 4 wt. %, about 1 to about 3 wt. %, about 1 to about 2 wt. %, about 1 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

Additionally, the skin care compositions typically comprise one or more peptide(s). The one or more peptides may comprise a plurality of peptides, such as 2, 3, 4, 5, 6, 7, 8, 9, 10 peptides, or any range formed therefrom. For example, the skin care composition may comprise a from 2 to 10 peptides, 2 to 8 peptides, 2 to 7 peptides, 2 to 6 peptides, 2 to 5 peptides, 2 to 4 peptides; from 3 to 10 peptides, 3 to 8 peptides, 3 to 7 peptides, 3 to 6 peptides, 3 to 5 peptides, 3 or 4 peptides; from 4 to 10 peptides, 4 to 8 peptides, 4 to 7 peptides, 4 to 6 peptides, 4 or 5 peptides; from 5 to 10 peptides, 5 to 8 peptides, 5 to 7 peptides, 5 or 6 peptides; from 6 to 10 peptides, 6 to 8 peptides, 6 or 7 peptides, or any range or subrange formed therefrom. In at least one embodiment, the skin care composition comprises 2 or more, 3 or more, 4 or more, 5 or more, 6 or more, 7 or more, or 8 or more peptides.

The one or more peptide(s) may comprise dipeptide diaminobutyroyl benzylamide diacetate, acetyl tetrapeptide-11, acetyl tetrapeptide-9, palmitoyl tripeptide-1, palmitoyl tetrapeptide-7, or a combination of two or more thereof. In some embodiments, the skin care composition comprises or consists of dipeptide diaminobutyroyl benzylamide diacetate, acetyl tetrapeptide-11, acetyl tetrapeptide-9, palmitoyl tripeptide-1, and palmitoyl tetrapeptide-7. The one or more peptide(s) may comprise or be selected from dipeptides. In at least one embodiment, the one or more peptide(s) comprises or consists of dipeptide diaminobutyroyl benzylamide diacetate.

The one or more peptide(s) may be present in the skin care composition in an amount up to about 1 wt. %, based on the total weight of the skin care composition. In some instances, the skin care composition may include peptide(s) in an amount up to about 0.7 wt. %, up to about 0.4 wt. %, up to about 0.2 wt. %, up to about 0.1 wt. %, or up to about 0.05 wt. %; from about 0.001 to about 1 wt. %; from about 0.001 to about 0.7 wt. %, about 0.001 to about 0.4 wt. %, about 0.001 to about 0.2 wt. %, about 0.001 to about 0.1 wt. %, about 0.001 to about 0.05 wt. %, about 0.001 to about 0.01 wt. %; from about 0.005 to about 0.7 wt. %, about 0.005 to about 0.4 wt. %, about 0.005 to about 0.2 wt. %, about 0.005 to about 0.1 wt. %; from about 0.01 to about 1 wt. %, about 0.01 to about 0.7 wt. %, about 0.01 to about 0.4 wt. %, about 0.01 to about 0.2 wt. %, about 0.01 to about 0.1 wt. %; from about 0.1 to about 1 wt. %, about 0.1 to about 0.7 wt. %, about 0.1 to about 0.4 wt. %, about 0.1 to about 0.2 wt. %; from about 0.2 to about 0.7 wt. %, about 0.2 to about 0.4 wt. %, about 0.4 to about 1 wt. %, about 0.4 to about 0.7 wt. %, or any range or subrange thereof, based on the total weight of the skin care composition.

In addition to a hyaluronic acid and/or a salt thereof, a vitamin E, and a peptide, the skin care composition typically comprises an extract comprising one or more of silybum marianum extract or pseudoalteromonas ferment extract. In at least one embodiment, the extract is in the form of an extract solution consisting essentially of or consisting of silybum marianum extract, pseudoalteromonas ferment extract, or a combination thereof along with a carrier. In at least one embodiment, the extract consists essentially of or consists of silybum marianum extract, pseudoalteromonas ferment extract, or a combination thereof. In some preferrable embodiments, the skin care composition comprises both silybum marianum extract and pseudoalteromonas ferment extract. By way of a non-limiting example, the oral care composition may have an extract component comprising silybum marianum extract and/or pseudoalteromonas ferment extract, wherein the total amount of the silybum marianum extract and/or pseudoalteromonas ferment extract is about 90 wt. % or more, about 95 wt. % or more, about 98 wt. % or more, or about 99 wt. % or more, relative to the total weight of the extract component.

The one or more extract(s) may be present in the skin care composition in an amount up to about 1 wt. %, based on the total weight of the skin care composition. In some instances, the skin care composition may include extracts(s) in an amount up to about 0.7 wt. %, up to about 0.4 wt. %, up to about 0.2 wt. %, up to about 0.1 wt. %, or up to about 0.05 wt. %; from about 0.001 to about 1 wt. %; from about 0.001 to about 0.7 wt. %, about 0.001 to about 0.4 wt. %, about 0.001 to about 0.2 wt. %, about 0.001 to about 0.1 wt. %, about 0.001 to about 0.05 wt. %, about 0.001 to about 0.01 wt. %; from about 0.005 to about 0.7 wt. %, about 0.005 to about 0.4 wt. %, about 0.005 to about 0.2 wt. %, about 0.005 to about 0.1 wt. %; from about 0.01 to about 1 wt. %, about 0.01 to about 0.7 wt. %, about 0.01 to about 0.4 wt. %, about 0.01 to about 0.2 wt. %, about 0.01 to about 0.1 wt. %; from about 0.1 to about 1 wt. %, about 0.1 to about 0.7 wt. %, about 0.1 to about 0.4 wt. %, about 0.1 to about 0.2 wt. %; from about 0.2 to about 0.7 wt. %, about 0.2 to about 0.4 wt. %, about 0.4 to about 1 wt. %, about 0.4 to about 0.7 wt. %, or any range or subrange thereof, based on the total weight of the skin care composition.

The skin care composition may include one or more polyol, e.g., in an amount from about 4 to about 22 wt. %, based on the total weight of the skin care composition. For instance, the skin care composition may include polyol(s) in an amount from about 4 to about 18 wt. %, about 4 to about 14 wt. %, about 4 to about 10 wt. %, about 4 to about 8 wt. %; from about 7 to about 22 wt. %, about 7 to about 18 wt. %, about 7 to about 14 wt. %, about 7 to about 10 wt. %; from about 10 to about 22 wt. %, about 10 to about 18 wt. %, about 10 to about 14 wt. %; from about 13 to about 22 wt. %, about 13 to about 18 wt. %; from about 16 to about 22 wt. %, about 16 to about 20 wt. %, about 19 to about 22 wt. %, or any range or subrange thereof, based on the total weight of the skin care composition.

The polyol(s) may be chosen from glycols or compounds with numerous hydroxyl groups. The term “polyol” should be understood as meaning, within the meaning of the present disclosure, an organic molecule comprising at least two free hydroxyl groups. The polyols of the skin care composition may be glycols or compounds with numerous hydroxyl groups. In some cases, the one or more polyols is/are selected from the group consisting of C₂-C₃₂ polyols. The one or more polyols may have from 2 to 32 carbon atoms, from 3 to 16 carbon atoms, or from 3 to 12 carbon atoms.

Polyols that may be included in the skin care composition, in certain instances, include ethylene glycol, propylene glycol, butylene glycol, caprylyl glycol hexylene glycol, glycerin, diglycerin, diethylene glycol, and dipropylene glycol, and mixtures thereof. In some cases, the polyol is propylene glycol and/or caprylyl glycol. In some further cases, the skin care composition comprises at least propylene glycol, and optionally one or more polyols other than propylene glycol, such as glycerin and/or hexanediol.

Non-limiting examples of polyols that may, optionally, be included in the skin care composition include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a combination of two or more thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or are chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and a mixture thereof. Non-limiting examples of polyols that may be included in the skin care composition, in certain instances, include ethylene glycol, propylene glycol, butylene glycol, hexylene glycol, diglycerin, diethylene glycol, and dipropylene glycol, and a combination of two or more thereof.

In certain embodiments, the skin care composition comprises two or more polyols. The plurality of polyols may comprise glycerin and one or more glycol. For example, the skin care composition may comprise a plurality of the polyols, wherein the plurality of the polyols comprise glycerin and one or more glycol selected from the group consisting of ethylene glycol, propylene glycol, butylene glycol, caprylyl glycol hexylene glycol, diethylene glycol, dipropylene glycol, and a combination of two or more thereof. In at least one embodiment, the skin care composition comprises propylene glycol and glycerin. In some instances, the polyols consist of propylene glycol and glycerin.

The skin care compositions may, in some preferred embodiments, have a polyol system comprising propylene glycol and glycerin in a weight ratio of the amount of propylene glycol to the amount of glycerin of about 2:1 to about 1:3. For instance, the skin care composition may have a weight ratio of the amount of propylene glycol to the amount of glycerin of about 2:1 to about 1:3, about 1:1 to about 1:3; from about 1:1.25 to about 1:3, about 1:1.5 to about 1:3, about 1:1.75 to about 1:3, about 1:2 to about 1:3; from about 1:1 to about 1:2.5, about 1:1.25 to about 1:2.5, about 1:1.5 to about 1:2.5, about 1:1.75 to about 1:2.5, about 1:2 to about 1:2; from about 1:1 to about 1:2, about 1:1.25 to about 1:2, about 1:1.5 to about 1:2, about 1:1.75 to about 1:2; from about 1:1 to about 1:1.5, about 1:1.25 to about 1:1.5, about 1:1 to about 1:1.25, or any range or subrange thereof.

The skin care compositions may optionally include one or more humectants. The humectant(s) may be selected from the polyols disclosed herein. Examples of humectants that may be incorporated in certain embodiments include triethylene glycol, tripropylene glycol, propylene glycol, caprylyl glycol, glycerin, polyethylene glycols, polypropylene glycol, glycerin, sorbitol, hexylene glycol, butylene glycol, hyaluronic acid, 1,2 hexanediol, saccharide isomerate and a combination of two or more thereof. In some cases, the one or more humectants comprise at least one of glycerin, caprylyl glycol, propylene glycol, or polyethylene glycol and, optionally, one or more additional humectants. Humectants can reduce evaporation and also contribute towards preservation by lowering water activity and can also impart desirable sweetness or flavor to compositions. In at least one embodiment, the one or more humectant comprises glycerin.

The skin care composition may include one or more nonionic surfactant in an amount that may vary, but may be from about 0.1 to about 5 wt. %, based on the total weight of the skin care composition. In some instances, the skin care composition includes nonionic surfactant(s) in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

The skin care composition may include one or more nonionic surfactant(s) selected from pegylated castor oil(s), polyglycerol ester(s), poloxamer(s), fatty alcohol ethoxylate(s), alkylphenol ethoxylate(s), tertiary amine oxide(s), glucoside(s), and a combination of two or more thereof. In at least one embodiment, the only surfactants in the skin care composition are nonionic surfactants selected from pegylated castor oil(s) and polyglycerol esters.

The pegylated castor oils may have a pegylated chain of about 10 to about 80. In some embodiments, the pegylated castor oils may have a pegylated chain of about 10 to about 80, about 10 to about 70, about 10 to about 60, about 10 to about 50, about 10 to about 40; from about 20 to about 80, about 20 to about 70, about 20 to about 60, about 20 to about 50, about 20 to about 40; from about 40 to about 80, about 40 to about 70, about 40 to about 60, about 60 to about 80, or any range or subrange thereof. In at least one embodiment, the skin care composition comprises one or more nonionic surfactant, including PEG-40 hydrogenated castor oil.

Additional examples of nonionic surfactants that are worth mentioning include long chain alkyl glucosides having C₈-C₂₂ alkyl groups; coconut fatty acid monoethanolamides such as cocamide MEA; coconut fatty acid diethanolamides, fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates; fatty acid alkylolamides, (fatty acid amide polyethylene glycols); N-alkyl-, N-alkoxypolyhydroxy fatty acid amides; sucrose esters; sorbitol esters; polyglycol ethers; and combinations of two or more thereof.

The skin care composition may, optionally, include one or more amphoteric surfactants. In some instances, the skin care composition includes amphoteric surfactant(s) in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

Amphoteric surfactants include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate. Examples of such compounds include sodium 3-dodecyaminopropionate, sodium 3-dodecylaminopropane sulfonate, N-alkyl taurines and N-higher alkyl aspartic acids.

Additional examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine, sulfobetaines such as coco dimethyl sulfopropyl betaine, stearyl dimethyl sulfopropyl betaine, amido betaines, amidosulfobetaines and the like. Betaines having a long chain alkyl group, particularly coco, may be useful as are those that include an amido groups such as the cocamidopropyl and cocoamidoethyl betaines. The amphoteric surfactant(s) may be in the form of a salt.

The skin care composition may include one or more silicone surfactant(s). The amount of silicone surfactant(s) present in the skin care composition may be from about 0.1 to about 5 wt. %, based on the total weight of the skin care composition. For example, the total amount of one or more silicone surfactant(s) in the skin care composition may be from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

The silicone surfactant may comprise or consist of anionic silicone surfactants. For instance, the silicone surfactant(s) may comprise methylsilanol mannuronate, hydroxyethyl acetomonium PG-dimethicone, lauryl polyglyceryl polydimethylsiloxyethyl dimethicone, or a combination of two or more thereof. The skin care composition may include or be free of cationic surfactants.

The skin care composition typically includes water in an amount of about 45 wt. % or more, based on the total weight of the skin care composition. For example, the total amount of water in the skin care composition may be from about 45 to about 94 wt. %, about 50 to about 94 wt. %, about 55 to about 94 wt. %, about 60 to about 94 wt. %, about 65 to about 94 wt. %, about 70 to about 94 wt. %, about 75 to about 94 wt. %, about 80 to about 94 wt. %; from about 45 to about 90 wt. %, about 50 to about 90 wt. %, about 55 to about 90 wt. %, about 60 to about 90 wt. %, about 65 to about 90 wt. %, about 70 to about 90 wt. %, about 75 to about 90 wt. %, about 80 to about 90 wt. %; from about 45 to about 86 wt. %, about 50 to about 86 wt. %, about 55 to about 86 wt. %, about 60 to about 86 wt. %, about 65 to about 86 wt. %, about 70 to about 86 wt. %, about 75 to about 86 wt. %, about 80 to about 86 wt. %; from about 45 to about 82 wt. %, about 50 to about 82 wt. %, about 55 to about 82 wt. %, about 60 to about 82 wt. %, about 65 to about 82 wt. %, about 70 to about 82 wt. %, about 75 to about 82 wt. %; from about 45 to about 78 wt. %, about 50 to about 78 wt. %, about 55 to about 78 wt. %, about 60 to about 78 wt. %, about 65 to about 78 wt. %, about 70 to about 78 wt. %; from about 45 to about 73 wt. %, about 50 to about 73 wt. %, about 55 to about 73 wt. %, about 60 to about 73 wt. %; from about 45 to about 68 wt. %, about 50 to about 68 wt. %, about 55 to about 68 wt. %; from about 45 to about 63 wt. %, about 50 to about 63 wt. %, or any range or subrange thereof, based on the total weight of the skin care composition.

Without being limited to any particular theories, it is believed that the skin care compositions may provide enhanced and/or additional benefits by incorporating certain amino acids, vitamin B, and/or pyrrolidone carboxylic acid and/or salts thereof in certain amounts and ratios. For instance, the addition of pyrrolidone carboxylic acid and/or sodium PCA and/or Niacinamide/Vitamin B3 in combination with both vitamin E (e.g., alpha-tocopherol) and a thiourea derivative of histidine (e.g., ergothioneine) may unexpectedly enhance and/or provide additional benefits when in combination with components of the skin care compositions disclosed herein.

In some embodiments, the skin care composition includes a thiourea derivative of histidine. The thiourea derivative of histidine may comprise ergothioneine or, in some embodiments, consists of ergothioneine. The thiourea derivative of histidine (e.g., ergothioneine) may be present in an amount from about 0.001 to about 2 wt. %, e.g., from 0.001 to about 2 wt. %, about 0.001 to about 1.5 wt. %, about 0.001 to about 1.25 wt. %, about 0.001 to about 1 wt. %, about 0.001 to about 0.5 wt. %, about 0.001 to about 0.1 wt. %, about 0.001 to about 0.05 wt. %, about 0.001 to about 0.01 wt. %, about 0.001 to about 0.009 wt. %, about 0.001 to about 0.008 wt. %, about 0.001 to about 0.0075 wt. %, about 0.001 to about 0.007 wt. %, about 0.001 to about 0.0065 wt. %, about 0.001 to about 0.006 wt. %, about 0.001 to about 0.0055 wt. %, about 0.001 to about 0.005 wt. %, about 0.001 to about 0.0045 wt. %, about 0.001 to about 0.004 wt. %, about 0.001 to about 0.0035 wt. %, about 0.001 to about 0.003 wt. %, about 0.001 to about 0.0025 wt. %, about 0.001 to about 0.002 wt. %, about 0.001 to about 0.0015 wt. %, or any range or subrange thereof, based on the total weight of the composition (e.g., wherein the weight refers to the active weight of thiourea derivative of histidine or EGT). In some embodiments, the thiourea derivative of histidine (e.g., ergothioneine) is present in an amount of about 0.0015 wt. %, about 0.002 wt. %, about 0.0025 wt. %, about 0.003%, about 0.0035 wt. %, about 0.004 wt. %, about 0.0045 wt. %, about 0.005 wt. %, about 0.0055 wt. %, about 0.006 wt. %, about 0.0065 wt. %, about 0.007 wt. %, about 0.0075 wt. %, about 0.008 wt. %, about 0.009 wt. %, about 0.01 wt. %, about 0.05 wt. %, about 0.1 wt. %, about 0.5 wt. %, about 1 wt. %, or any range or subrange formed therefrom, based on the total weight of the composition (e.g., wherein the weight refers to the active weight of thiourea derivative of histidine or EGT).

The skin care composition may include one or more vitamin B. The vitamin B may comprise or consist of vitamin B3 and/or derivatives thereof. The formula of the vitamin B3 may comprise the following structure:

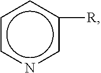

wherein R is —CONH₂ (i.e., niacinamide), —COOH (i.e., nicotinic acid) or —CH₂OH (i.e., nicotinyl alcohol); a derivative thereof, and/or a salt thereof.

The vitamin B3 and/or a derivative thereof may be selected from the group consisting of nicotinic acid esters, non-vasodilating esters of nicotinic acid, nicotinyl amino acids, nicotinyl alcohol esters of carboxylic acids (e.g., straight-chain or branched chain, cyclic or acyclic, saturated or unsaturated (including aromatic), substituted or unsubstituted nicotinic acid N-oxide, niacinamide N-oxide, tocopherol nicotinate, inositol hexanicotinate, and a combination of two or more theroef. Examples of derivatives of niacinamide include nicotinyl amino acids, derived, e.g., from the reaction of an activated nicotinic acid compound (e.g., nicotinic acid azide or nicotinyl chloride) with an amino acid, and nicotinyl alcohol esters of organic carboxylic acids (e.g., C₁-C₁₈). Examples of such derivatives include nicotinuric acid and nicotinyl hydroxamic acid.

Additional derivatives of niacinamide includes nicotinyl alcohol esters, such as nicotinyl alcohol esters of the carboxylic acids, salicylic acid, acetic acid, glycolic acid, palmitic acid and the like. Further examples of derivatives of niacinamide include 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine, and combinations thereof. In at least one embodiment, the vitamin B comprises or consists of niacinamide.

The amount of vitamin B present in the skin care composition may be from about 0.1 to about 5 wt. %, based on the total weight of the skin care composition. For instance, the skin care composition may comprise a total amount of vitamin B of about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 0.7 to about 5 wt. %, about 0.7 to about 4 wt. %, about 0.7 to about 3 wt. %, about 0.7 to about 2 wt. %, about 0.7 to about 1 wt. %; from about 1 to about 5 wt. %, about 1 to about 4 wt. %, about 1 to about 3 wt. %, about 1 to about 2 wt. %, about 1 to about 1.5 wt. %, about 1 to about 1.25 wt. %; from about 1.25 to about 5 wt. %, about 1.25 to about 4 wt. %, about 1.25 to about 3 wt. %, about 1.25 to about 2 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition. In some embodiments, the amount of vitamin B in the skin care composition is about 4.75 wt. %, about 4.5 wt. %, about 4.25 wt. %, about 4 wt. %, about 3.75 wt. %, about 3.5 wt. %, about 3.25 wt. %, about 3 wt. %, about 2.75 wt. %, about 2.5 wt. %, about 2.25 wt. %, about 2 wt. %, about 1.75 wt. %, about 1.5 wt. %, about 1.25 wt. %, about 1 wt. %, about 0.5 wt. %, or any range formed therefrom, based on the total weight of the composition.

Pyrrolidone carboxylic acid and/or a salt thereof may be incorporated into certain embodiments of the skin care composition. The pyrrolidone carboxylic acid and/or a salt thereof may be in the form of a salt, such as sodium PCA. In some embodiments, the total amount of pyrrolidone carboxylic acid and/or a salt thereof consists of pyrrolidone carboxylic acid or consists of non-incidental amounts of pyrrolidone carboxylic acid. In other embodiments, the total amount of pyrrolidone carboxylic acid and/or a salt thereof consists of salts of pyrrolidone carboxylic acid (e.g., sodium PCA) or non-incidental amounts of salts of pyrrolidone carboxylic acid. The pyrrolidone carboxylic acid and/or a salt thereof may comprise L-pyrrolidone carboxylic acid and/or a salt thereof.

In some embodiments, the skin care composition comprises pyrrolidone carboxylic acid and/or salt(s) thereof in an amount from about 0.1 to about 5 wt. %, based on the total weight of the skin care composition. For example, the total amount of the pyrrolidone carboxylic acid and/or salt(s) thereof present in the skin care composition may be from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 0.7 to about 5 wt. %, about 0.7 to about 4 wt. %, about 0.7 to about 3 wt. %, about 0.7 to about 2 wt. %, about 0.7 to about 1 wt. %; from about 1 to about 5 wt. %, about 1 to about 4 wt. %, about 1 to about 3 wt. %, about 1 to about 2 wt. %, about 1 to about 1.5 wt. %, about 1 to about 1.25 wt. %; from about 1.25 to about 5 wt. %, about 1.25 to about 4 wt. %, about 1.25 to about 3 wt. %, about 1.25 to about 2 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

The skin care compositions may comprise one or more amino acids. The skin care composition may have an amino acid component comprising a plurality of amino acids. The amino acids may be selected from arginine, lysine, serine, citrulline, ornithine, creatine, histidine, diaminobutanoic acid, diaminoproprionic acid, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, a salt thereof, or a combination of two or more thereof.

In certain embodiments, the skin care composition comprises an amino acid component comprising ergothioneine and at least one additional amino acid. For example, in certain preferred embodiments, the amino acid component comprises ergothioneine and at least one amino acid selected from the group consisting of taurine, arginine, glycine, or a combination of two or more thereof. In at least one preferred embodiment, the amino acid component comprises ergothioneine, taurine, arginine, and glycine. The amino acid component may consist essentially of or consist of ergothioneine, taurine, arginine, and glycine in some instances.

In some embodiments, the disclosure provides a personal care composition comprising taurine, arginine and glycine, wherein taurine is in free or physiologically acceptable salt form. The term taurine also encompasses derivatives of taurine such as its salts and esters as well as its complexes, conjugates and the like, and specifically includes biogenic taurine derivatives such as chloramine/taurine complexes, as well as synthetic and semi-synthetic derivatives. Unless otherwise specified, weight ratios of taurine described herein are based on the neutral form of taurine having molecular formula C₂H₇NSO₃.

Arginine and glycine can each be in free or physiologically acceptable salt form. Arginine is preferably L-arginine, Arginine Hydrochloride or L-Arginine Hydrochloride. Unless otherwise specified, weight ratios of arginine and glycine described herein are based on their neutral forms having molecular formula of C₆H₁₄N₄O₂, and C₂H₅NO₂, respectively.

The skin care composition may include one or more of amino acid(s) in an amount from about 0.01 to about 5 wt. %, based on the total weight of the skin care composition. In some embodiments, the skin care composition may include an amino acid in amount from about 0.01 to about 2 wt. %, about 0.01 to about 1.7 wt. %, about 0.01 to about 1.4 wt. %, about 0.01 to about 1.2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %, about 0.01 to about 0.05 wt. %; from about 0.05 to about 3 wt. %, about 0.05 to about 2 wt. %, about 0.05 to about 1.7 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 3 wt. %, about 0.1 to about 2 wt. %, about 0.1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 3 wt. %, about 1 to about 2 wt. %, about 1 to about 1.7 wt. %, about 1 to about 1.4 wt. %, about 1 to about 1.2 wt. %; from about 1.5 to about 3 wt. %, about 1.5 to about 2 wt. %, about 2 to about 3 wt. %, or any range or subrange thereof, based on the total weight of the skin care composition.

As discussed above, the skin care composition may include an amino acid component comprising a plurality of amino acids. The total amount of amino acid(s) (e.g., the amount of the amino acid component) present in the skin care composition may be from about 0.1 to about 7 wt. %, based on the total weight of the skin care composition. For example, the total amount of amino acid(s) in the skin care composition may be from about 0.1 to about 6 wt. %, about 0.1 to about 5 wt. %, about 0.1 to about 3 wt. %, about 0.1 to about 2 wt. %, about 0.1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 7 wt. %, about 0.3 to about 5 wt. %, about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 7 wt. %, about 0.6 to about 5 wt. %, about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 7 wt. %, about 1 to about 5 wt. %, about 1 to about 3 wt. %, about 1 to about 2 wt. %, about 1 to about 1.7 wt. %, about 1 to about 1.4 wt. %, about 1 to about 1.2 wt. %; from about 1.5 to about 7 wt. %, about 1.5 to about 5 wt. %, about 1.5 to about 3 wt. %, about 1.5 to about 2 wt. %, from about 2 to about 7 wt. %, about 2 to about 5 wt. %, about 2 to about 3 wt. %; from about 3 to about 7 wt. %, about 3 to 5 wt. %, about 4 to about 7 wt. % about 4 to 6 wt. %, or any range or subrange thereof, based on the total weight of the skin care composition.

In certain preferred embodiments, the skin care composition comprises taurine, arginine and glycine. The skin care composition may be specifically formulated to have taurine, arginine, and glycine in a weight ratio of taurine to arginine to glycine (taurine:arginine:glycine) of (1.5-69):(0.5-40):(0.5-1.5), respectively. For instance, the skin care composition may be formulated to have a weight ratio of the total amount of taurine to the total amount of arginine to the total amount of glycine (taurine:arginine:glycine) from about 85:45:1 to about 45:24:1. In some embodiments, the skin care composition has a weight ratio of the total amount of taurine to the total amount of arginine to the total amount of glycine from about 80:42:1 to about 50:30:1; from about 75:40:1 to about 55:29:1; from about 70:37:1 to about 60:31:1, or any range or subrange thereof. In at least one embodiment, the skin care composition comprises a weight ratio of the total amount of taurine to the total amount of arginine to the total amount of glycine of about (65):(34):(1), respectively.

In at least one embodiment, the skin care composition comprises:
taurine, arginine, and glycine, wherein the skin care composition has a weight ratio of the total amount of taurine to the total amount of arginine to the total amount of glycine (taurine:arginine:glycine) of (1.5-69):(0.5-40):(0.5-1.5), respectively (e.g., in a weight ratio of about (65):(34):(1), respectively);
ergothioneine (EGT);
alpha-tocopherol; and
Vitamin B3/niacinamide.

In at least one embodiment, the skin care composition comprises:
taurine, arginine and glycine, wherein the skin care composition has a weight ratio of the total amount of taurine to the total amount of arginine to the total amount of glycine (taurine:arginine:glycine) of (1.5-69):(0.5-40):(0.5-1.5), respectively (e.g., in a weight ratio of about (65):(34):(1), respectively);
ergothioneine (EGT);
alpha-tocopherol; and
pyrrolidone carboxylic acid and/or a salt thereof (e.g., sodium PCA).

In at least one embodiment, the skin care composition comprises:
taurine, arginine and glycine, wherein the skin care composition has a weight ratio of the total amount of taurine to the total amount of arginine to the total amount of glycine (taurine:arginine:glycine) of (1.5-69):(0.5-40):(0.5-1.5), respectively (e.g., in a weight ratio of about (65):(34):(1), respectively);
ergothioneine (EGT);
alpha-tocopherol;
pyrrolidone carboxylic acid and/or a salt thereof (e.g., sodium PCA); and
Vitamin B3/Niacinamide.

In at least one embodiment, the skin care composition comprises:
ergothioneine (EGT);
alpha-tocopherol; and
pyrrolidone carboxylic acid and/or a salt thereof (e.g., sodium PCA),
wherein the skin care composition has a weight ratio of the total amount of alpha-tocopherol to the total amount of EGT to the total amount of pyrrolidone carboxylic acid and/or a salt thereof (alpha-tocopherol:EGT:pyrrolidone carboxylic acid and/or a salt thereof) of (0.5-2):(0.5-20):(0.5-20), respectively (e.g., 1:1:1) (e.g., 1:1:2) (e.g., 1:10:10), wherein the weight ratios are given with respect to the raw material weight % of the ingredients.

In at least one embodiment, the skin care composition comprises:
ergothioneine (EGT);
alpha-tocopherol; and
niacinamide,
wherein the alpha-tocopherol, the EGT, and the niacinamide are in a weight ratio of the total amount of alpha-tocopherol to the total amount of EGT to the total amount of niacinamide of (0.5-2):(0.5-20):(0.5-20), respectively (e.g., 1:1:1) (e.g., 1:1:2) (e.g., 1:1:3) (e.g., 1:2:1) (e.g., 1:3:1) (e.g., 2:1:1) and (e.g., 3:1:1), wherein the weight ratios are given with respect to the raw material weight % of the ingredients.

In at least one embodiment, the skin care composition comprises:
ergothioneine (EGT);
alpha-tocopherol; and
pyrrolidone carboxylic acid and/or a salt thereof (e.g., sodium PCA),
wherein the skin care composition has a weight ratio of the total amount of alpha-tocopherol to the total amount of EGT to the total amount of pyrrolidone carboxylic acid and/or a salt thereof (alpha-tocopherol:EGT:pyrrolidone carboxylic acid and/or a salt thereof) of (50-250):(0.5-20):(50-250), respectively (e.g., 50:1:50, 50:1:100, 50:1:150:50:1:200, 50:1:250, 100:1:50, 100:1:100, 100:1:150:100:1:200, 100:1:250, 150:1:50, 150:1:100, 150:1:150:150:1:200, 150:1:250, 200:1:50, 200:1:100, 200:1:150, 200:1:200, 200:1:250, 250:1:50, 250:1:100, 250:1:150:250:1:200, 250:1:250, or any range or subrange thereof), wherein the weight ratios are based on the active material weight % of the ingredients.

In at least one embodiment, the skin care composition comprises:
ergothioneine (EGT);
alpha-tocopherol; and
niacinamide
wherein the skin care composition has a weight ratio of the total amount of alpha-tocopherol to the total amount of EGT to the total amount of niacinamide (alpha-tocopherol:EGT:niacinamide) of (50-250):(0.5-20):(100-500), respectively (e.g., 50:1:100, 50:1:150, 50:1:200, 50:1:250, 50:1:300, 50:1:350, 50:1:400, 50:1:450, 50:1:500, 100:1:100, 100:1:150, 100:1:200, 100:1:250, 100:1:300, 100:1:350, 100:1:400, 100:1:450, 100:1:500, 150:1:100, 150:1:150, 150:1:200, 150:1:250, 150:1:300, 150:1:350, 150:1:400, 150:1:450, 150:1:500, 200:1:100, 200:1:150, 200:1:200, 200:1:250, 200:1:300, 200:1:350, 200:1:400, 200:1:450, 200:1:500, 250:1:100, 250:1:150, 250:1:200, 250:1:250, 250:1:300, 250:1:350, 250:1:400, 250:1:450, 250:1:500, or any range or subrange thereof), wherein the weight ratios are based on the active material weight % of the ingredients.

In at least one embodiment, the skin care composition comprises:
ergothioneine (EGT);
alpha-tocopherol;
niacinamide; and
pyrrolidone carboxylic acid and/or a salt thereof (e.g., sodium PCA).

In at least one embodiment, the skin care composition has a weight ratio of taurine, arginine and glycine: niacinamide: carnosine: alpha-tocopherol is about 2:1:1:1 or 2:2:1:1, respectively.

In at least one embodiment, the skin care composition, the skin care composition is formulated to have a weight ratio of the total amount of alpha-tocopherol to the total amount of EGT of about 6:1 to about 1:6. For instance, the skin care composition may have a weight ratio of the total amount of alpha-tocopherol to the total amount of EGT from about 6:1 to about 1:6, about 5:1 to about 1:6, about 4:1 to about 1:6, about 3:1 to about 1:6, about 2:1 to about 1:6, about 1:1 to about 1:6; from about 6:1 to about 1:5, about 5:1 to about 1:5, about 4:1 to about 1:5, about 3:1 to about 1:5, about 2:1 to about 1:5, about 1:1 to about 1:5; from about 6:1 to about 1:4, about 5:1 to about 1:4, about 4:1 to about 1:4, about 3:1 to about 1:4, about 2:1 to about 1:4, about 1:1 to about 1:4; from about 6:1 to about 1:3, about 5:1 to about 1:3, about 4:1 to about 1:3, about 3:1 to about 1:3, about 2:1 to about 1:3, about 1:1 to about 1:3; from about 6:1 to about 1:2, about 5:1 to about 1:2, about 4:1 to about 1:2, about 3:1 to about 1:2, about 2:1 to about 1:2, about 1:1 to about 1:2; from about 6:1 to about 1:1, about 5:1 to about 1:1, about 4:1 to about 1:1, about 3:1 to about 1:1, about 2:1 to about 1:1, about 1:1, or any range or subrange thereof.

Additionally or alternatively, the skin care compositions may be formulated to have a weight ratio of the total amount of alpha-tocopherol to the total amount of niacinamide and/or a weight ratio of the total amount of EGT to the total amount of niacinamide of about 1:1 to about 1:11. In some embodiments, the skin care compositions has a weight ratio of the total amount of the alpha-tocopherol to the total amount of the niacinamide and/or a weight ratio of the total amount of EGT to the total amount of niacinamide from about 1:1 to about 1:11, about 1:1 to about 1:9, about 1:1 to about 1:7, about 1:1 to about 1:6, about 1:1 to about 1:5, about 1:1 to about 1:4, about 1:1 to about 1:3; from about 1:2 to about 1:11, about 1:2 to about 1:9, about 1:2 to about 1:7, about 1:2 to about 1:6, about 1:2 to about 1:5, about 1:2 to about 1:4, about 1:2 to about 1:3; from about 1:3 to about 1:11, about 1:3 to about 1:9, about 1:3 to about 1:7, about 1:3 to about 1:6, about 1:3 to about 1:5, about 1:3 to about 1:4, about 1:3, or any range or subrange thereof.

Additionally or alternatively, the skin care compositions may be formulated to have a weight ratio of the total amount of alpha-tocopherol to the total amount of pyrrolidone carboxylic acid and/or a salt thereof and/or a weight ratio of the total amount of EGT to the total amount of pyrrolidone carboxylic acid and/or a salt thereof of about 1:1 to about 1:11. In some embodiments, the skin care compositions has a weight ratio of the total amount of alpha-tocopherol to the total amount of pyrrolidone carboxylic acid and/or a salt thereof and/or a weight ratio of the total amount of EGT to the total amount of pyrrolidone carboxylic acid and/or a salt thereof from about 1:1 to about 1:11, about 1:1 to about 1:9, about 1:1 to about 1:7, about 1:1 to about 1:6, about 1:1 to about 1:5, about 1:1 to about 1:4, about 1:1 to about 1:3; from about 1:2 to about 1:11, about 1:2 to about 1:9, about 1:2 to about 1:7, about 1:2 to about 1:6, about 1:2 to about 1:5, about 1:2 to about 1:4, about 1:2 to about 1:3; from about 1:3 to about 1:11, about 1:3 to about 1:9, about 1:3 to about 1:7, about 1:3 to about 1:6, about 1:3 to about 1:5, about 1:3 to about 1:4, about 1:3, or any range or subrange thereof.

In some embodiments the skin care compositions comprise a weight ratio of the total amount of alpha-tocopherol to the total amount of EGT to the total amount of pyrrolidone carboxylic acid and/or a salt thereof (alpha-tocopherol:EGT:pyrrolidone carboxylic acid and/or a salt thereof) of (0.5-2):(0.5-20):(1-20), respectively (e.g., 1:1:1) (e.g., 1:1:2) (e.g., 1:10:10), where the weight ratios are based upon the weights of the ingredients relative to the total weight of the composition. In other embodiments, skin care composition comprises an alpha-tocopherol, EGT, and sodium PCA in a weight ratio of the total amount of alpha-tocopherol to the total amount of EGT to the total amount of sodium PCA of (50-250):(0.5-20):(50-250), respectively (e.g., (200:1:200), where the weight ratios are based on the active material weight % of the ingredients relative to the total weight of the composition. In certain embodiments, the skin care composition may be formulated to have a weight ratio of the total amount of alpha-tocopherol to the total amount of EGT to the total amount of pyrrolidone carboxylic acid and/or a salt thereof (alpha-tocopherol:EGT:pyrrolidone carboxylic acid and/or a salt thereof) from about 1:2:6 to about 1:0.5:1.5, from about 1:1.5:4 to about 1:0.75:2; about 1:1.1:3.3 to about 1:0.9:2.7.

In certain embodiments, the skin care composition comprises a weight ratio of the total amount of alpha-tocopherol to the total amount of EGT to the total amount of niacinamide (alpha-tocopherol:EGT:niacinamide) in a weight ratio of (0.5-2):(1-20):(1-20), respectively (e.g., 1:1:1) (e.g., 1:1:2) (e.g., 1:1:3), where the weight ratios are based upon the raw material weight % of the ingredients relative to the total weight of the composition. In certain embodiments, the skin care composition may be formulated to have a weight ratio of the total amount of alpha-tocopherol to the total amount of EGT to the total amount of niacinamide (alpha-tocopherol:EGT:niacinamide) from about 1:2:6 to about 1:0.5:1.5, from about 1:1.5:4 to about 1:0.75:2; about 1:1.1:3.3 to about 1:0.9:2.7. In further embodiments, the skin care composition comprises a weight ratio of the total amount of alpha-tocopherol to the total amount of EGT to the total amount of niacinamide (alpha-tocopherol:EGT:niacinamide) in a weight ratio of (100-250):(0.5-5):(200-600) (e.g., 200:1:400), where the weight ratios are based on the active material weight % of the ingredients.

In at least one embodiment, the skin care composition comprises:
niacinamide (e.g., from 0.5%-5% by wt. of the composition) (e.g., about 3% by wt.);
alpha-tocopherol (e.g., from 0.1%-2% by wt. of the composition) (e.g., about 0.5% by wt.); and
ergothioneine (EGT) (e.g., from 0.001-0.005% by wt. of the composition) (e.g., about 0.0025% by wt.).

In at least one embodiment, the skin care composition comprises:
ascorbyl glucoside (e.g., from 0.5%-5% by wt. of the composition) (e.g., about 2% by wt.), wherein the ascorbyl glucoside is present in an amount preferably from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; about 3 to about 5 wt. %, about 3 to about 4 wt. %, or any range or subrange thereof, based on the total weight of the skin care composition; and
astaxanthin (e.g., from 0.001%-0.05% by wt. of the composition) (e.g., about 0.01% by wt.).

In at least one embodiment, the skin care composition comprises:
ergothioneine (EGT);
alpha-tocopherol;
niacinamide (vitamin B);
pyrrolidone carboxylic acid and/or a salt thereof (e.g., sodium PCA);
hyaluronic acid (e.g., having a MW of 50 kDa);
magnesium sulfate; and
nicotinamide adenine dinucleotide (NAD).

The skin care composition may include a fatty component comprising one or more fatty compound. The fatty component may comprise a fatty ester, fatty alcohol, a fatty ether, an oil, a wax, or a combination of two or more thereof. The fatty component may be present in an amount from about 0.1 to about 20 wt. %, based on the total weight of the skin care composition. In some instances, the fatty compound(s) are present in an amount of about 0.1 to about 18 wt. %, about 0.1 to about 16 wt. %, about 0.1 to about 14 wt. %, about 0.1 to about 12 wt. %, about 0.1 to about 10 wt. %, about 0.1 to about 8 wt. %, about 0.1 to about 7 wt. %, about 0.1 to about 6 wt. %, about 0.1 to about 5 wt. %; about 0.5 to 20 wt. %, about 0.5 to about 18 wt. %, about 0.5 to about 16 wt. %, about 0.5 to about 14 wt. %, about 0.5 to about 12 wt. %, about 0.5 to about 10 wt. %, about 0.5 to about 8 wt. %, about 0.5 to about 7 wt. %, about 0.5 to about wt. %, about 0.5 to about 5 wt. %; about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 7 wt. %, about 1 to about 6 wt. %, about 1 to about 5 wt. %; about 2 to about 20 wt. %, about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 7 wt. %, about 2 to about 6 wt. %, about 2 to about 5 wt. %; about 3 to about 20 wt. %, about 3 to about 18 wt. %, about 3 to about 16 wt. %, about 3 to about 14 wt. %, about 3 to about 12 wt. %, about 3 to about 10 wt. %, about 3 to about 8 wt. %, about 3 to about 7 wt. %, about 3 to about 6 wt. %, about 3 to about 5 wt. %; about 4 to about 20 wt. %, about 4 to about 18 wt. %, about 4 to about 16 wt. %, about 4 to about 14 wt. %, about 4 to about 12 wt. %, about 4 to about 10 wt. %, about 4 to about 8 wt. % about 4 to about 7 wt. %, about 4 to about 6 wt. %, about 4 to about 5 wt. %; about 5 to about 20 wt. %, about 5 to about 18 wt. %, about 5 to about 16 wt. %, about 5 to about 14 wt. %, about 5 to about 12 wt. %, about 5 to about 10 wt. %, or about 5 to about 8 wt. %, about 5 to about 7 wt. %, or about 5 to about 6 wt. %, including all ranges and sub-ranges thereof, based on the total weight of the skin care composition.

The fatty compounds may include or be chosen from one or more oil(s). The term “oil” means any fatty substance that is in liquid form at room temperature (24-25° C.) and at atmospheric pressure (760 mmHg). The oil may be a hydrocarbon-based oil, such as a saturated hydrocarbon oil, an unsaturated hydrocarbon oil, lipids, triglycerides, a natural oil, and/or a synthetic oil. The one or more oil(s) may be synthetic oils and/or plant-based oils. For instance, the oil may be a synthetic oil, such as a mineral oil and/or an alkane (paraffins). Light (low viscosity) highly branched hydrocarbon oils may also be suitable in some instances. In at least one embodiment, the skin care composition includes an oil, such as isododecane, isodecane, decane, dodecane, or a combination of two or more thereof.

In some embodiments, the oil is chosen from plant-based or vegetable-based oils, such as coconut oil, soybean oil, canola oil, rapeseed oil, corn oil, cottonseed oil, olive oil, palm oil, peanut oil, safflower oil, sesame oil, sunflower seed oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and a mixture thereof. In at least one embodiment, the oil(s) are selected from the fatty component comprises a plant-based oil chosen from sunflower oil, corn oil, rosa moschata seed oil, soybean oil, marrow oil, hydrogenated vegetable oil, grapeseed oil, sesame seed oil, hazelnut oil, apricot oil, macadamia oil, arara oil, castor oil, avocado oil, caprylic/capric acid triglycerides, jojoba oil and shea butter oil, and a combination of two or more thereof.

The oil may be volatile or nonvolatile, cyclic, linear or branched silicone oils, which are unmodified or modified with organic groups. The silicone oil may be chosen from polydialkylsiloxanes, such as polydimethylsiloxanes (PDMS), and liquid polyorganosiloxanes. In some cases, the silicone oil may be selected from polydialkylsiloxanes, octamethylcyclotetrasiloxane, tetra(trimethylsilyl)pentaerythritol, octamethylcyclotetrasiloxane, polydiphenylsiloxanes, polyalkylarylsiloxanes, or a combination of two or more thereof. Skin compatible oils may be silicone based, for example, linear and cyclic polydimethyl siloxane, organo functional silicones (alkyl and alkyl aryl), and amino silicones.

Additional skin compatible oils include a range of liquid hydrocarbons, for example, linear and branched oils such as liquid paraffin, squalene, squalane, mineral oil, low viscosity synthetic hydrocarbons, such as polyalphaolefins and polybutene. In at least one preferred embodiment, the oil comprises or consists of a dimethicone.

The fatty compounds may, in some instances, include or be chosen from one or more waxes. Non-limiting examples of waxes in this category include, e.g., synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as, sunflower seed (helianthus annuus), carnauba, candelilla, ouricury or japan wax or cork fiber or sugarcane waxes), or a mixture thereof. In some embodiments, the fatty component comprises a wax chosen from rhus verniciflua peel wax, butyrospermum parkii (Shea) butter, or a combination thereof.

The fatty component comprises a fatty ester chosen from stearyl stearate, dihydroabietyl behenate, octyldodecyl behenate, isocetyl behenate, isostearyl lactate, lauryl lactate, linoleyl lactate, oleyl lactate, isostearyl octanoate, isocetyl octanoate, octyl octanoate, decyl oleate, isocetyl isostearate, isocetyl laurate, isocetyl stearate, isodecyl octanoate, isodecyl oleate, isononyl isononanoate, isostearyl palmitate, methyl acetyl ricinoleate, octyl isononanoate, 2-ethylhexyl isononanoate, octyldodecyl erucate, oleyl erucate, ethyl palmitate, isopropyl palmitate, such as 2-ethylhexyl palmitate, 2-octyldecyl palmitate, alkyl myristates such as isopropyl 2-octyldodecyl myristate, isobutyl stearate, 2-hexyldecyl laurate, isocetyl stearate, octyldodecyl stearoyl stearate, or a combination thereof. In some embodiments, the fatty ester is selected from include glycerol fatty esters, sorbitan fatty esters, ethoxylated fatty esters, synthetic esters, and a combination of two or more thereof.

The fatty component may comprise one or more liquid fatty alcohol(s). The fatty alcohol may be chosen from linear or branched, saturated or unsaturated alcohols, preferably unsaturated or branched alcohols, comprising from 6 to 40 carbon atoms, preferably from 8 to 30 carbon atoms. Examples of fatty alcohols include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and a combination of two or more thereof.

One or more thickening agent(s) may be included in the skin care composition. The amount of thickening agent(s) in the skin care composition may be from about 0.05 to about 5 wt. %, based on the total weight of the skin care composition. In some embodiments, the total amount of thickening agent(s) in the skin care composition is from about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.7 wt. %; from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 1 to about 5 wt. %, about 1 to about 4 wt. %, about 1 to about 3 wt. %, about 1 to about 2 wt. %, about 1 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

The thickening agent(s) may be chosen from carboxylic acid polymers; crosslinked polyacrylate polymers; polyacrylamide polymers; gums, polysaccharides, and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

Non-limiting examples of carboxylic acid polymers include crosslinked carboxylic acid polymers containing one or more monomers derived from acrylic acid, substituted acrylic acids, and salts and esters of these acrylic acids and the substituted acrylic acids, such as carbomers. For example, the thickening agent(s) may include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, or a combination of two or more thereof. In certain embodiments, the one or more thickening agent may comprise or consist of natural gum(s), such as those chosen from xanthan gum, guar gum, biosaccharide gum, cellulose, acacia seneca gum, sclerotium gum, agarose, pechtin, gellan gum, hyaluronic acid, and a combination of two or more thereof. In some embodiments, the thickening agent comprises polyglycerides including agar, agarose, pectin, guars, or a combination of two or more thereof.

In certain cases, the skin care composition may be formulated to have thickening agents selected from thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (e.g., carboxymethyl cellulose, methyl cellulose, methylhydroxypropyl cellulose, hydroxyethyl cellulose, hydroxypropyl cellulose, cellulose sulfate sodium salt), and/or starch derivatives (e.g., starch oxide, dialdehyde starch, dextrin, British gum, acetyl starch, starch phosphate, carboxymethyl starch, hydroxyethyl starch, hydroxypropyl starch). In some embodiments, the thickening agent(s) are selected from among hydroxypropyl methylcellulose, polyethylene glycols (PEGs), polyacrylic acids, cross-linked homopolymer of acrylic acid, acrylates of C₁₀₋₃₀ alkyl acrylate crosspolymer, and a combination of two or more thereof.

The skin care compositions may include one or more additional vitamins, such as vitamin C, vitamin D, and vitamin K. The additional vitamins may be individually present in the skin care composition in an amount of about 0.01 to about 5 wt. %, based on the total weight of the skin care composition. In some instances, the amount of additional vitamins present in the skin care composition are from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the skin care composition.

As used herein, “vitamin C” may be ascorbic acid or derivatives thereof. Ascorbic acid exists as two enantiomers commonly denoted “l” (for “levo”) and “d” (for “dextro”). The “l” isomer is the one most often encountered. Ascorbic acid is also referred to as L(+)-ascorbic acid or 1-ascorbic acid. The ascorbic acid derivatives may be or include, but are not limited to, L-ascorbic acid, calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof.

As used herein, “vitamin D” refers to a group of fat-soluble secosteroids responsible for increasing intestinal absorption of calcium, magnesium, and phosphate, and many other biological effects. The two major forms are vitamin D₂ or ergocalciferol, and vitamin D₃ or cholecalciferol. Vitamin D includes vitamin D₁ (mixture of molecular compounds of ergocalciferol with lumisterol), vitamin D₂ (ergocalciferol), vitamin D₃ (cholecalciferol), vitamin D₄ (22-dihydroergocalciferol), and vitamin D₅ (sitocalciferol).

As used herein, “vitamin K” is a group of compounds with a common chemical structure of 2-methyl-1,4-naphthoquinone. Vitamin K plays a role in blood clotting, bone metabolism, and regulating blood calcium levels. Vitamin K includes vitamin K₁ (phylloquinone) and vitamin K₂ (menaquinone). Vitamin K₂ typically has unsaturated isoprenyl side chains and are designated as MK-4 through MK-13, based on the length of their side chain.

Optionally, solvents may be included in the skin care composition. The solvent(s) may comprise: water-soluble alcohols, such as C₂₋₈ alcohols including ethanol; glycols including propylene glycol, dipropylene glycol, tripropylene glycol and mixtures thereof; glycerides including mono-, di-, and triglycerides; medium to long chain organic acids, alcohols and esters; surfactants including emulsifying and dispersing agents; amino acids including glycine; structurants including thickening agents and gelling agents, for example polymers, silicates and silicon dioxide; emollients; fragrances; and colorants including dyes and pigments.

Additional ingredients may be present in the skin care composition. These include ingredients to thicken, preserve, emulsify, add fragrance, adjust the pH, and color. For example, the skin care composition may include one or more emulsifier, pH adjuster, colorant, fragrance, preservative, antioxidant, emollients, or the like. The skin care compositions may include any of the following additional ingredients in an amount of from about 0.01 to about 5 wt. %. In some instances, the amount of additional ingredient present in the skin care composition, individually or cumulatively, is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the skin care composition.

The skin care compositions may contain one or more additional ingredient that is an emollient. Non-volatile emollients are preferable. Classes of non-volatile emollients include non-silicone and silicone emollients. Non-volatile, non-silicone emollients include C₁₂₋₁₅ alkyl benzoate. The non-volatile silicone material can be a polyethersiloxane, polyalkyarylsiloxane, or polyethersiloxane copolymer. An illustrative non-volatile silicone material is phenyl trimethicone. Examples include, but are not limited to, PPG-14 butyl ether, PPG-3 myristyl ether, secondary alcohol ethoxylates, stearyl alcohol, stearic acid and salts thereof, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, acetylated lanolin alcohols, acetylated lanolin, petrolatum, isopropyl ester of lanolin, fatty acids, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide, or a combination of two or more thereof.

The skin care compositions may optionally include one or more or more additional ingredient that is an antioxidant. For example, one or more antioxidants may be added to the composition to act as ingredient protectants and for maintenance of long-term stability of the composition. Examples of optional antioxidants include, but are not limited to, citric acid, butylated hydroxytoluene, pentaerythrityl tetra-di-t-butyl hydroxyhydrocinnamate. In at least one embodiment, the antioxidants are selected from flavonoids, phytoalexins, carnosine, ascorbic acid, tocotrienol, a lipoic acid, a carotenoid, melatonin, and a combination of two or more thereof.

Non-limiting examples of fragrances and perfumes include odor compounds selected from: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl)propionaldehyde, ethylvanillin, heliotropin, hexylcinnamaldehyde, amylcinnamaldehyde, 2-methyl-2-(isopropylphenyl)propionaldehyde, coumarin, γ-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, tert-butylcyclohexyl acetate, and a combination of two or more thereof.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl)cyclo-hexanol acetate, benzyl acetate, and eugenol. The fragrances or perfumes can be used as single substances or in a mixture with one another.

The skin care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes.

The colorants may be present in an amount ranging from about 0.0001 to about 0.4 wt. %, including all percentages and subranges therebetween, based on the total weight of the skin care composition. In some embodiments, the colorants may be present in an amount ranging from about 0.0001 to about 4 wt. %, including all percentages and subranges therebetween, based on the total weight of the skin care composition. In certain embodiments, in order to prevent ingredients from separating, emulsifiers may be added.

In other embodiments, the skin care composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5% by weight); sequestrants, for example, tetrasodium EDTA, and/or their equivalents (0 to 2% by weight); biocides, for example, triclosan (2,4,4′-trichloro-2′-hydroxydiphenyl ether), DMDM hydantoin, formaldehyde and/or imidazolidinyl urea, and/or their equivalents (0 to 2% by weight); organic acids, for example, citric acid and/or formic acid and/or their equivalents (0 to 2% by weight); viscosity modifiers (0 to 2% by weight); fragrances and/or perfumes (0 to 5% by weight); preservatives, for example, phenoxyethanol, formaldehyde solution, parabens, pentanediol or sorbic acid (0 to 2% by weight); pearlizing agents, for example, glycol distearic esters, such as ethylene glycol distearate, but also fatty acid monoglycol esters (0 to 3% by weight); stabilizers, for example, metal salts of fatty acids, such as e.g. magnesium stearate, aluminum stearate and/or zinc stearate (0 to 2% by a weight); and dyes and pigments that are approved and suitable for cosmetic purposes. In some embodiments, the preservatives may comprise one or more of citric acid (and/or sodium citrate), benzoic acid (and/or sodium benzoate), benzotriazolyl codecyl P-cresol, and/or salts thereof.

The skin care composition may include one or more pH adjusters to increase or decrease the overall pH of the skin care composition. For example, one or more acids may be included to decrease the pH of the skin care composition. Examples of suitable acids for decreasing the pH of the skin care composition include, but are not limited to, citric acid, acetic acid, and the like. The skin care composition may include one or more bases, such as sodium hydroxide, potassium hydroxide and the like, to increase the pH of the skin care composition. Additional or alternative acids and bases that are suitable for adjusting the pH of the skin care composition are readily known to one of ordinary skill in the art.

The amount of the pH adjuster in the skin care composition may be based on the desired pH of the final skin care composition and/or product. For example, the total amount of the pH adjuster may range from about 0.05 to about 20 wt. %, based on the total weight of the skin care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the skin care composition.

In certain embodiments, the skin care composition has a pH of from about 4 to about 10.0. In other embodiments, the skin care composition has a pH of from about 4.5 to about 8.0. In some embodiments, the skin care composition has a pH of from about 5 to about 7.5. In other embodiments, the skin care composition has a pH of from about 5.5 to about 7. In further embodiments, the skin care composition has a pH of about 6 to about 7.

In some embodiments, the additional ingredient is selected from carnosine, cica (asiaticoside), magnesium sulfate, saccharide isomerate, a keto-carotenoid (e.g., astaxanthin), nicotinamide adenine dinucleotide (NAD), and a combination of two or more thereof.

Anti-inflammatory agents may be included as additional ingredients in the skin care composition. The anti-inflammatory agent(s) may be selected from the group consisting of: dexamethasone, prednisolone, corticosterone, budesonide, sulfasalazine, mesalamine, cetirizine, diphenylhydramine, antipyrine, methyl salicylate, loratadine, thymol, carvacrol, bisabolol, allantoin, eucalyptol, phenazone, propyphenazone, a nonsteroidal anti-inflammatory drug NSAID, and any combinations thereof.

Additionally or alternatively, the skin care composition may include one or more additional agent selected from anti-itch agents. Examples of anti-itch agents methyl sulphonyl methane, sodium bicarbonate, calamine, allantoin, kaolin, peppermint, tea tree oil, camphor, menthol, and/or hydrocortisone.

In some embodiments, the skin care compositions may further comprise additional ingredients selected from non-ionic block copolymers. Examples of non-ionic block copolymers include from: poloxamer 338, poloxamer 407, poloxamer, 237, poloxamer, 217, poloxamer 124, poloxamer 184, poloxamer 185, or a combination of two or more thereof.

The skin care composition may optionally include delivery vesicle (e.g., a multilamellar vesicle (“MLV”). For instance, the skin care composition may have one or more ingredients that are encapsulated in a delivery vesicle, such as a multilamellar vesicle. The multilamellar vesicle may comprises liposome structuring agents comprising polyglyceryl-10 dioleate and at least one of polyglyceryl-10 dilinoleate or polyglyceryl-10 dipalmitate in a weight ratio of polyglyceryl-10 dioleate to the at least one of polyglyceryl-10 dilinoleate or polyglyceryl-10 dipalmitate of from 300:1 to 5:1. In some instances, the multilamellar vesicle comprises one or more ingredients selected from: polyglyceryl-10 dilinoleate, polyglyceryl-10 dipalmitate, polyglyceryl-10 dioleate, Caprylic/Capric Triglyceride, and combinations thereof. The skin care composition may comprise delivery vesicle when in the form of a using a transdermal injection (e.g., mesotherapy composition).

Suspending agents such as silicas or clays such as bentonite, montmorillonite or hectorite, including those available under the trademark Bentone can also be employed to thicken liquid skin care compositions. The composition can be thickened with non-polymeric organic gellants, including selected dibenzylidene alditols (e.g., dibenzylidene sorbitol).

The skin care compositions of the disclosure may be manufactured using methods known in the art. Typically, the ingredients are combined and optionally heated where components need to be melted. The components are mixed. Desirably, volatile materials such as fragrant materials are incorporated in the composition in the latter stages of a mixing cycle in order to avoid volatilization thereof. After mixing, the composition may be poured directly into the dispensers and the container capped to preserve the product until use.

The present disclosure provides, in another aspect, a skin care composition for use in inhibiting, treating or reducing damage to the skin caused reactive oxygen species. The skin care composition may be any of the skin care compositions disclosed herein.

The present disclosure provides, in another aspect, a method of inhibiting or reducing damage to the skin caused reactive oxygen, wherein the method comprises administering to skin an effective amount of any of skin care compositions disclosed herein. The method may comprise using a skin care composition, where the skin care composition (e.g., any of the skin care compositions disclosed herein) is delivered via a transdermal injection. Additionally or alternatively, the method may comprise using a skin care composition, where the skin care composition (e.g., any of the skin care compositions disclosed herein) is topically delivered to the skin using a serum, a cream, a moisturizer, a mask, a cleanser, oil, salve, lotion, gel, ointment, paste a facial make-up a toner, an essence and a balm (e.g., lip balm).

### EXAMPLES

### Example 1

A non-limiting, example skin care composition (Example Composition A) was prepared in the form of a serum in accordance with aspects of the invention. The formulation of Example Composition A is shown in Table 1.

TABLE 1

| | Ex. A |
|---|---|
| INCI Compound Name | (wt. %) |
| Water | 78-84 |
| Propanediol | 5.1-5.5 |
| Glycerin | 5-5.4 |
| Pentylene Glycol and 1,2-Hexanediol | 1.1-1.3 |
| Caprylic/Capric Triglyceride | 0.1-0.3 |
| Polyglyceryl-10 Dioleate | 0.1-0.3 |
| PEG-40 Hydrogenated Castor Oil | 1.9-2.2 |
| Triethyl Citrate | 1.9-2.2 |
| Xanthan Gum | 0.05-0.1 |
| Tocopherol | 0.001-0.01 |
| Tocopheryl Acetate | 0.1-0.3 |
| Sodium PCA | 0.1-0.3 |
| Ergothioneine | 0.001-0.005 |
| Niacinamide | 0.4-0.6 |
| Carnosine | 0.1-0.3 |
| Sodium Hyaluronate | 0.1-0.3 |
| Hydrolyzed Hyaluronic Acid | 0.01-0.1 |
| Arginine | 0.1-0.3 |
| Adenosine | 0.01-0.05 |
| Polyglyceryl-10 Dipalmitate | 0.03-0.05 |
| Silybum Marianum Extract | 0.01-0.04 |
| Glyceryl Caprylate | 0.01-0.05 |
| Dipeptide Diaminobutyroyl Benzylamide Diacetate | 0.01-0.03 |
| Mannitol | 0.1-0.3 |
| Glycine | 0.001-0.005 |
| Pseudoalteromonas Ferment Extract | 0.001-0.003 |
| Magnesium Sulfate | 0.001-0.003 |
| Nicotinamide Adenine Dinucleotide | 0.001-0.003 |
| Taurine | 0.1-0.4 |
| Colorants | 0.0001-0.001 |
| Fragrance | 0.3 |
| Preservative (Citric Acid, Sodium Citrate, | 1.2 |
| Benzoic Acid, Sodium Benzoate, and | |
| Benzotriazolyl Dodecyl P-Cresol) | |

### Example 2

Example Composition A was clinically evaluated by performing a cutometric assessment, an in vivo clinical assessment, a topographical assessment, and a self-evaluation assessment. Thirty-two female volunteers of ages from 40 to 65 years with healthy skin having wrinkles, fine lines, a lack of firmness, crow's feet of a grade of 2 or higher, as graded according to the scale disclosed in Doublet, ATLAS DU VIEILLISSEMENT CUTANE were selected for the evaluation of Example Composition A.

The volunteers underwent a washout period of about two weeks before applying Example Composition A. After the washout period, Example Composition A was applied twice a day, specifically once in the morning and once in the evening, for 28 days. The volunteers were evaluated before application of Example Composition A, on day 7 of application of Example Composition A, and on day 28 of application of Example Composition A.

### Cutometric Assessment

For the cutometric assessment, each volunteer underwent a 20-minute period of acclimatization to the air-conditioned room at a temperature of 21±1.5° C. prior to the cutometric measurements. The cutometric measurements were performed on the skin of the volunteer's temple. The same area of skin was used for each of the cutometric measurements.

The procedure for the cutometric measurements included the steps of: application of a constant negative pressure, withdrawal of the strain, and a relaxation period. Inside the cutometer, a penetration depth was determined by a non-contact optical measuring system. The optical measuring system contained a light source and a light receptor, as well as two prisms facing each other, which projected the light from transmitter to receptor. The light intensity varied due to the penetration depth of the skin. The resistance of the skin to the negative pressure (firmness) and its ability to return into its original position (elasticity) were obtained and displayed as curves (penetration depth in mm/time) in real time during the measurement. The cutometric assessment allows for the measurement of elastic and mechanical properties of the skin surface and enables objective quantification of skin aging. The experimental conditions for the cutometric measurements were: diameter probe: 2 mm; pressure: 300 mbar; on-time: 2 s off-time: 2 s repetition (cycles number): 1.

The cutometric measurements evaluated the following skin properties: maximum extensibility (final deformation); immediate extensibility (purely elastic component); visco-elastic extensibility (plasticity); immediate elastic recovery; elastic deformation rate (immediate elastic recovery/immediate extensibility); elastic recovery rate (immediate elastic recovery/maximum extensibility); and visco-elasticity rate (visco-elastic extensibility/immediate extensibility).

Table 2 provides a summary of the results of the cutometric measurements at the initial evaluation prior to the application of Example Composition A, at day 7 day of application of Example Composition A, and at day 28 day of application of Example Composition A.

TABLE 2

| | Initial | Evaluation | Evaluation |
|---|---|---|---|
| | Evaluation | at Day 7 | at Day 28 |
| Maximum | 0.28 (±0.052) | 0.264 (±0.052) | 0.259 (±0.046) |
| Extensibility (mm) | | | |
| Immediate | 0.182 (±0.042) | 0.169 (±0.042) | 0.166 (±0.033) |
| Extensibility (mm) | | | |
| Visco-Elastic | 0.098 (±0.015) | 0.094 (±0.014) | 0.093 (±0.018) |
| Extensibility (mm) | | | |
| Immediate Elastic | 0.067 (±0.015) | 0.064 (±0.015) | 0.061 (±0.012) |
| Recovery (mm) | | | |
| Elastic Deformation | 0.373 (±0.075) | 0.385 (±0.07) | 0.375 (±0.082) |
| Rate (no units) | | | |
| Elastic Recovery | 0.241 (±0.047) | 0.245 (±0.04) | 0.239 (±0.05) |
| Rate (no units) | | | |
| Visco-Elasticity Rate | 0.548 (±0.082) | 0.571 (±0.084) | 0.565 (±0.096) |
| (no units) | | | |

Table 3 provides a summary of the average change as a percentage of the volunteer's skin from the initial evaluation (“T0”) to the evaluation at day 7 (“T7”) and from the initial evaluation to the evaluation at day 28 (“T28”).

TABLE 3

| | ΔT_{T7-T0} | ΔT_{T28-T0} |
|---|---|---|
| | ((T7 − T0)/T0) | ((T28 − T0)/T0) |
| Maximum Extensibility | −5.6% | −7.3% |
| Immediate Extensibility | −6.9% | −8.6% |
| Visco-Elastic Extensibility | −3.2% | −5.0% |
| Immediate Elastic Recovery | −4.1% | −8.8% |
| Elastic Deformation Rate | 3.1% | 0.3% |
| Elastic Recovery Rate | 1.5% | −0.8% |
| Visco-Elasticity Rate | 4.2% | 3.0% |

The statistical analysis of the results of the cutometric measurements shows a significant decrease, on average, for the following parameters: maximum extensibility of −5.6% and −7.3% at day 7 and day 28 respectively; immediate extensibility of −6.9% and −8.6% at day 7 and day 28respectively; visco-elastic extensibility of −5.0% at day 28; and immediate elastic recovery of −8.8% at day 28. Notably, the significant decrease in the maximum extensibility parameter observed at day 7 (an average of −5.6%) and at day 28 (an average of −7.3%) shows the desirable tensor effect on the face skin achieved by Example Composition A.

In vivo Clinical Assessment

For the in vivo clinical assessment, the skin of each volunteer was evaluated by an expert at the initial evaluation prior to the application of Example Composition A, at day 7 day of application of Example Composition A, and at day 28 day of application of Example Composition A. Specifically, the skin of each volunteer was evaluated by assessing the main wrinkle depth on the crow's feet of the volunteer, the skin complexion homogeneity, the skin luminosity, the skin radiance, and the skin firmness. The expert carried out the evaluation of the parameter using a dedicated scale with lower numerical values referring to less intensity of such parameter and higher numerical values referring to more intensity of such parameter.

A summary of the results of in vivo clinical assessment of the crow's feet of the volunteer, the skin complexion homogeneity, the skin luminosity, the skin radiance, and the skin firmness is shown in Table 4 below.

TABLE 4

| | Initial | Evaluation | Evaluation |
|---|---|---|---|
| | Evaluation | at Day 7 | at Day 28 |
| Main Wrinkle Depth of Crow's Feet | 3.5 (±1.3) | 3.3 (±1.3) | 3.2 (±1.4) |
| Skin Complexion Homogeneity | 3.2 (±1.1) | 3.4 (±1) | 3.6 (±1) |
| Skin Luminosity | 2.6 (±0.7) | 2.5 (±0.6) | 2.6 (±0.6) |
| Skin Firmness | 2.9 (±0.9) | 2.8 (±1) | 2.8 (±1) |
| Index of Skin Radiance | 2.9 (±0.8) | 3 (±0.7) | 3.1(±0.8) |

Table 5 shows the average percentage change of the volunteer's skin from the initial evaluation (“T0”) to the evaluation at day 7 (“T7”) and from the initial evaluation to the evaluation at day 28 (“T28”).

TABLE 5

| | ΔT_{T7-T0} | ΔT_{T28-T0} |
|---|---|---|
| | ((T7 − T0)/T0) | ((T28 − T0)/T0) |
| Main Wrinkle Depth of Crow's Feet | −4% | −8.5% |
| Skin Complexion Homogeneity | 7.6% | 12.9% |
| Skin Luminosity | −3.8% | 0.2% |
| Skin Firmness | −3.9% | 6.4% |
| Index of Skin Radiance | 2.5% | 7.2% |

In comparison with the baseline, the statistical analysis shows after 7 and 28 days of application of Example Composition A, a significant improvement was exhibited in the main wrinkle depth of the crow's feet with respectively −0.1 and −0.3 grade out of 8 (i.e., respectively −4.0% and −8.5%) and the skin complexion homogeneity with respectively +0.2 and +0.4 out of 10 (i.e., respectively +7.6% and +12.9%). Additionally, a significant increase in the index of radiance was demonstrated at day 28 after application of Example Composition A with an improvement of 7.2%.

### Topographical Assessment

The skin topography (crow's feet wrinkles) and skin texture of the volunteers was assessed by means of cross polarized and diffuse light photographs. Specifically, high resolution ¾ face photographs were obtained for each volunteer, in completely reproducible lighting conditions. Photographs were carried out with a high-resolution camera. The lens used was a Nikkor 60 mm equipped with a filter. Lighting was provided by two flashlights. The flash heads were fitted with filter slots to hold polarizing gel. To obtain cross polarized light, the filter on the lens of the camera was positioned at 90° compared to the polarization of the filters of the flashes. The polarized light, emitted by the flashes and reflected by the skin of the face at the moment the photo is taken, was cut by the camera filter. These reflections do not appear on the photograph when it is obtained. To obtain diffuse light photos, the filter on the lens of the camera was turned to 45° compared to the position for cross polarized light.

The use of different filters allow for the characterization mapping of the topography of the skin. The surface and the density of the detected wrinkles on a delimited area was then calculated using software. The total surface of wrinkles (a topographic parameter expressed in pixels²) for each volunteer was determined. A decrease in this parameter corresponds to an anti-wrinkles effect. Additionally, the surface of detected roughness (texture parameter expressed in pixels²) was determined for each volunteer. A decrease in this parameter corresponds to a smoothing effect of the skin grain.

Table 6 provides a summary of the total surface of detected roughness and wrinkles observed at initial evaluations, at day 7 of application of Example Composition A, and at day 28 of application of Example Composition A.

TABLE 6

| | Initial | Evaluation | Evaluation |
|---|---|---|---|
| | Evaluation | at Day 7 | at Day 28 |
| Total surface of detected | 34104 | 33315 | 32082 |
| roughness (pixels ²) | (±17945) | (±17890) | (±17341) |
| Total surface of wrinkles/ | 13100 | 12702 | 11925 |
| roughness - Crow's feet | (±7030) | (±6540) | (±6101) |
| (pixels ²) | | | |

Table 7 provides the average change in the total surface of detected roughness and wrinkles observed from the initial evaluation (“T0”) to the evaluation at day 7 (“T7”) and from the initial evaluation to the evaluation at day 28 (“T28”).

TABLE 7

| | ΔT_{T7-T0} | ΔT_{T28-T0} |
|---|---|---|
| | ((T7 − T0)/T0) | ((T28 − T0)/T0) |
| Total surface of detected | −2.3% | −5.9% |
| roughness | | |
| Total surface of wrinkles/ | −3% | −9% |
| roughness - Crow's feet | | |

As shown in Table 6, the statistical analysis establishes a significant decrease in the total surface of detected roughness and in the total surface of wrinkles on the crow's feet at day 28 of application of Example Composition A as compared to the initial evaluation, with 5.9% and 9.0% average decreases, respectively.

### Self-Evaluation Assessment

The volunteers completed self-evaluations by completing a questionnaire before application of Example Composition A, at day 7 of application of Example Composition A, and at day 28 of application of Example Composition A. From the questionnaire completed on day 7,all 43 of the questions received a significantly favorable answer, with the skin being “hydrated” receiving a 97% of agreement by volunteers; and the skin being “more supple”, “the complexion is clarified”, and “the product visibly brightens the skin” receiving a 94% of agreement by volunteers. From the questionnaire completed on day 28, all 43 of the questions received a significantly favorable answer, with the skin being “hydrated” receiving 100% agreement by the volunteers; the skin being “nourished”, “toned” and “visibly firmer” receiving 97% of agreement by the volunteers; and the skin being “more supple”, “clarified”, “as lifted”, “smoother”, and “revitalized” receiving 94% of agreement by the volunteers. Additionally, the volunteers estimated that their wrinkles were visibly “reduced” and “smoothed,” with each receiving 91% and 94% agreement by volunteers, respectively.

### Example 3

Example Composition A was clinically evaluated by performing macrophotography to assess cell renewal. Specifically, dihydroxyacetone (DHA) was applied to the forearm of each volunteer of Example 2 using a DHA patch. DHA is widely used in self-tanning (e.g., sunless tanner) products as applying DHA to the skin makes the skin turn brown. This color arises because of DHA's interactions with amino acids. Without being limited to any particular theory, it is believed that DHA penetrates through the stratum corneum, possibly reaching the top layers of the stratum granulosum, and binds to the skin forming a DHA-skin complex (DHA-SC). Because the DHA-SC is an integral part of the skin surface, its substantivity is only limited by the natural desquamation of the skin or by physical removal of the corneocytes.

Measurements were performed on the area of the forearm receiving DHA and a similar area of forearm, which did not receive the DHA. Specifically, photographs of these two areas were carried out before DHA application (for a baseline), after application of the DHA but before application of Example Composition A, and at day 4, day 5, day 6, and day 7 of application of Example Composition A. For each day of application, Example Composition A was applied once in the morning and once in the evening to the forearm of the volunteers.

The absolute measurements were expressed in the form of L*a*b*, with L* representing the relative brightness from total black (L*=0) to total white (L*=100); a* representing the balance between red and green; and b* representing the balance between yellow and blue. Using the absolute measurements, the following parameter was calculated on each area: ΔE=√(ΔL²+Δa²+Δb²): difference in color observed on an area, between two times. ΔE is a measure of the visual difference between colors.

The ΔE parameter on day 5, 6, and 7 of application of Example Composition A showed that an improvement in cell renewal occurred in the area of skin receiving Example Composition A as compared to an area of skin not receiving Example Composition A.

### Example 4

Example Composition A was clinically evaluated in comparison to a mesotherapy composition (Comparative Composition 1) by performing an in vivo clinical assessment and a cutometric assessment. The list of ingredients for the mesotherapy composition is provided below:
Hyaluronic acid, non-crosslinked sodium hyaluronate, ascorbic acid, biotin, pantothenic acid, folic acid, inositol, Nicotinamide, pyridoxine, riboflavin, thiamine, tocopherol, retinol, vitamin B12, calcium chloride, potassium chloride, magnesium sulfate, sodium acetate, sodium chloride, sodium dihydrogen phosphate, deoxyadenosine, deoxycytidine, deoxyguanosine, deoxythymidine, 5-methyl-2-deoxycytidine, α-aminobutyric acid, alanine, arginine, asparagine, aspartic acid, cystine, glutamine, glutamic acid, glycine, histidine, hydroxyproline, isoleucine, leucine, lysine, methionine, ornithine, phenylalanine, proline, serine, taurine, threonine, tryptophane, tyrosine, and valine.

Fifty female volunteers with healthy skin having dull complexion, dehydrated skin, fine lines, wrinkles, and crow's feet of a grade of equal or less than 4, as graded according to the scale disclosed in Doublet, ATLAS DU VIEILLISSEMENT CUTANE. The volunteers were separated into two groups, with the first group receiving Comparative Composition 1 and the second group receiving Example Composition A once each day for 63 days. The volunteers were evaluated before application of the respective composition (“D0”), at day 21 of continued application of the respective composition (“D21”), at day 42 of continued application of the respective composition (“D42”), and day 63 of continued application of the respective composition (“D63”).

In vivo Clinical Assessment

For the in vivo clinical assessment, the skin of each volunteer was evaluated by an expert at the initial evaluation prior to the application of Example Composition A, at day 7 day of application of Example Composition A, and at day 28 day of application of Example Composition A. The skin of each volunteer was evaluated by an expert by assessing the main wrinkle depth on the crow's feet of the volunteer, the skin homogeneity, the visibility of undereye wrinkles, the skin radiance, and the smooth aspect of the skin. The expert carried out the evaluation of the respective parameters using dedicated scales with lower numerical values referring to less intensity of such parameter and higher numerical values referring to more intensity of such parameter.

The skin's hydration level was evaluated using a MoistureMeterD Compact devices, which is a portable moisture meter designed to measure localized water content over a specific area (expressed in %). The probe of the moisture meter generates a low-power 300 MHz microwave field exposed to the tissue. Part of the signal is absorbed and part is reflected to calculate a dielectric constant, which is directly proportional to the water content of the tissue being measured. The value obtained by the moisture meter increases with increasing water content.

Hydration level was measured on each volunteer's cheekbone right or left (according to the randomization) at D0, D21 (±48 h), D42 (±48 h) and D63 (±48 h). Raw data was completed directly in the eCRF by the assessor.

Summaries of the results of the in vivo clinical assessments are shown in Tables 8-13. Table 8 presents a summary of the effect on the skin's radiance provided by Example Composition A and Comparative Composition 1.

TABLE 8

| | | Comparative | Example |
|---|---|---|---|
| | | Composition | Composition |
| Complexion radiance | | 1 | A |
| Initial | Mean (sd) | 5.42 | 4.58 |
| Evaluation | | (0.81) | (1.03) |
| (D 0) | Group comparison | | p = |
| | vs Group 1 | | 0.007 |
| Day 21 | Mean (sd) | 6.12 | 5.12 |
| (D 21) | | (1.03) | (0.99) |
| | Group comparison | | p = |
| | vs Group 1 | | 0.476 |
| Δ_{D 21-D 0} | Mean (sd) of | 0.69 | 0.54 |
| | absolute difference | (0.47) | (0.58) |
| | Timepoints comparison | p < | p < |
| | vs D 0 | 0.001 | 0.001 |
| | Mean relative | 13% | 13% |
| | difference | | |
| Day 42 | Mean (sd) | 6.56 | 5.5 |
| (D 42) | | (0.87) | (1.06) |
| | Group comparison | | p = |
| | vs Group 1 | | 0.184 |
| Δ_{D 42-D 0} | Mean (sd) of | 1.12 | 0.83 |
| | absolute difference | (0.53) | (0.64) |
| | Timepoints comparison | p < | p < |
| | vs D 0 | 0.001 | 0.001 |
| | Mean relative | 21% | 19% |
| | difference | | |
| Day 63(D | Mean (sd) | 6.56 | 5.64 |
| 63) | | (0.87) | (1.04) |
| | Group comparison | | p = |
| | vs Group 1 | | 0.912 |
| Δ_{D 63-D 0} | Mean (sd) of | 1.12 | 1 |
| | absolute difference | (0.53) | (0.58) |
| | Timepoints comparison | p < | p < |
| | vs D 0 | 0.001 | 0.001 |
| | Mean relative | 21% | 23% |
| | difference | | |

*p is a measure of statistical significance.

A summary of the improvement on the crow's feet wrinkles provided by Example Composition A and Comparative Composition 1 is presented in Table 9.

TABLE 9

| | | Comparative | Example |
|---|---|---|---|
| | | Composition | Composition |
| Crow's Feet Wrinkles Appearance | | 1 | A |
| Initial | Mean (sd) | 2.43 | 2.44 |
| Evaluation | | (0.72) | (0.91) |
| (D 0) | Group comparison | / | p = |
| | vs Group1 | | 0.973 |
| Day 21 | Mean (sd) | 2.36 | 2.45 |
| (D 21) | | (0.69) | (0.9) |
| | Group comparison | / | p = |
| | vs Group1 | | 0.108 |
| Δ_{D 21-D 0} | Mean (sd) of | −0.07 | 0.02 |
| | absolute difference | (0.19) | (0.11) |
| | Timepoints comparison | p = | p = |
| | vs D 0 | 0.643 | 1 |
| | Mean relative | −2% | 1% |
| | difference | | |
| Day 42 | Mean (sd) | 2.3 | 2.33 |
| (D 42) | | (0.69) | (0.8) |
| | Group comparison | / | p = 0.019 |
| | vs Group1 | | |
| Δ_{D 42-D 0} | Mean (sd) of | −0.15 | −0.04 |
| | absolute difference | (0.16) | (0.2) |
| | Timepoints comparison | p < | p = |
| | vs D 0 | 0.001 | 0.969 |
| | Mean relative | −6% | 0% |
| | difference | | |
| Day 63 | Mean (sd) | 2.29 | 2.31 |
| (D 63) | | (0.69) | (0.84) |
| | Group comparison | / | p = |
| | vs Group1 | | 0.145 |
| Δ_{D 63-D 0} | Mean (sd) of | −0.16 | −0.1 |
| | absolute difference | (0.17) | (0.22) |
| | Timepoints comparison | p < | p = |
| | vs D 0 | 0.001 | 0.092 |
| | Mean relative | −6% | −3% |
| | difference | | |

*p is a measure of statistical significance.

Table 10 presents a summary of the improvement on the skin's homogeneity provided by Example Composition A and Comparative Composition 1.

TABLE 10

| | | Comparative | Example |
|---|---|---|---|
| | | Composition | Composition |
| Skin Homogeneity | | 1 | A |
| Initial | Mean (sd) | 4.77 | 4.15 |
| Evaluation | | (0.82) | (1.22) |
| (D 0) | Group comparison | | p = |
| | vs Group1 | | 0.102 |
| Day 21 | Mean (sd) | 5.04 | 4.65 |
| (D 21) | | (0.92) | (1.06) |
| | Group comparison v | | p = |
| | s Group1 | | 0.092 |
| Δ_{D 21-D 0} | Mean (sd) of | 0.27 | 0.5 |
| | absolute difference | (0.45) | (0.51) |
| | Timepoints comparison | p = | p < |
| | vs D 0 | 0.184 | 0.001 |
| | Mean relative | 6% | 15% |
| | difference | | |
| Day 42 | Mean (sd) | 5.16 | 4.88 |
| (D 42) | | (1.07) | (1.12) |
| | Group comparison | | p = |
| | vs Group1 | | 0.284 |
| Δ_{D 42-D 0} | Mean (sd) of | 0.36 | 0.67 |
| | absolute difference | (0.49) | (0.76) |
| | Timepoints comparison | p = | p < |
| | vs D 0 | 0.016 | 0.001 |
| | Mean relative | 7% | 20% |
| | difference | | |
| Day 63 | Mean (sd) | 5.24 | 4.64 |
| (D 63) | | (1.05) | (1.04) |
| | Group comparison | | p = |
| | vs Group1 | | 0.884 |
| Δ_{D 63-D 0} | Mean (sd) of absolute | 0.44 | 0.44 |
| | difference | (0.51) | (0.58) |
| | Timepoints comparison | p < | p < |
| | vs D 0 | 0.001 | 0.001 |
| | Mean relative | 9% | 14% |
| | difference | | |

*p is a measure of statistical significance.

A summary of the improvement on the smooth aspect of skin provided by Example Composition A and Comparative Composition 1 is presented in Table 11.

TABLE 11

| | | Comparative | Example |
|---|---|---|---|
| | | Composition | Composition |
| Smooth Aspect of the Skin | | 1 | A |
| Initial | Mean (sd) | 5.58 | 4.92 |
| Evaluation | | (0.76) | (1.41) |
| (D 0) | Group comparison | | p = |
| | vs Group1 | | 0.206 |
| Day 21 | Mean (sd) | 6 | 5.27 |
| (D 21) | | (0.94) | (1.19) |
| | Group comparison | | p = |
| | vs Group1 | | 0.093 |
| Δ_{D 21-D 0} | Mean (sd) of | 0.42 | 0.35 |
| | absolute difference | (0.7) | (0.49) |
| | Timepoints comparison | p = | p = |
| | vs D 0 | 0.017 | 0.12 |
| | Mean relative | 8% | 10% |
| | difference | | |
| Day 42 | Mean (sd) | 6.2 | 5.67 |
| (D 42) | | (1.08) | (1.34) |
| | Group comparison | | p = |
| | vs Group1 | | 0.885 |
| Δ_{D 42-D 0} | Mean (sd) of | 0.64 | 0.67 |
| | absolute difference | (0.64) | (0.64) |
| | Timepoints comparison | p < | p < |
| | vs D 0 | 0.001 | 0.001 |
| | Mean relative | 11% | 17% |
| | difference | | |
| Day 63 | Mean (sd) | 6.16 | 5.64 |
| (D 63) | | (1.14) | (1.41) |
| | Group comparison | | p = |
| | vs Group1 | | 0.672 |
| Δ_{D 63-D 0} | Mean (sd) of | 0.6 | 0.68 |
| | absolute difference | (0.71) | (0.69) |
| | Timepoints comparison | p < | p < |
| | vs D 0 | 0.001 | 0.001 |
| | Mean relative | 11% | 16% |
| | difference | | |

*p is a measure of statistical significance.

Table 12 presents a summary of the improvement on undereye wrinkles provided by Example Composition A and Comparative Composition 1.

TABLE 12

| | | Comparative | Example |
|---|---|---|---|
| Visibility of Wrinkles | | Composition | Composition |
| Underneath the Eyes | | 1 | A |
| Initial | Mean (sd) | 2.38 | 2.48 |
| Evaluation | | (0.66) | (0.84) |
| (D 0) | Group comparison | | p = |
| | vs Group1 | | 0.083 |
| Day 21 | Mean (sd) | 2.33 | 2.55 |
| (D 21) | | (0.57) | (0.79) |
| | Group comparison | | p = |
| | vs Group1 | | 0.992 |
| Δ_{D 21-D 0} | Mean (sd) of | −0.05 | 0.07 |
| | absolute difference | (0.25) | (0.24) |
| | Timepoints comparison | p = | p = |
| | vs D 0 | 0.999 | 0.238 |
| | Mean relative | 0% | 4% |
| | difference | | |
| Day 42 | Mean (sd) | 2.3 | 2.5 |
| (D 42) | | (0.66) | (0.8) |
| | Group comparison | | p = |
| | vs Group1 | | 0.043 |
| Δ_{D 42-D 0} | Mean (sd) of | −0.06 | 0.07 |
| | absolute difference | (0.35) | (0.26) |
| | Timepoints comparison | p = | p = |
| | vs D 0 | 0.989 | 0.975 |
| | Mean relative | −2% | 5% |
| | difference | | |
| Day 63 | Mean (sd) | 2.3 | 2.38 |
| (D 63) | | (0.66) | (0.82) |
| | Group comparison | | p = |
| | vs Group1 | | 0.494 |
| Δ_{D 63-D 0} | Mean (sd) of | −0.06 | −0.07 |
| | absolute difference | (0.35) | (0.39) |
| | Timepoints comparison | p = | p = |
| | vs D 0 | 0.989 | 0.963 |
| | Mean relative | −2% | −2% |
| | difference | | |

*p is a measure of statistical significance.

A summary of the improvement on the hydration level of skin provided by Example Composition A and Comparative Composition 1 is presented in Table 13.

TABLE 13

| | | Comparative | Example |
|---|---|---|---|
| | | Composition | Composition |
| Hydration Level | | 1 | A |
| Initial | Mean (sd) | 43.81 | 41.93 |
| Evaluation | | (3.32) | (3.76) |
| (D 0) | Group comparison | | p = |
| | vs Group 1 | | 0.674 |
| Day 21 | Mean (sd) | 43.6 | 43.58 |
| (D 21) | | (2.81) | (3.7) |
| | Group comparison | | p = 1 |
| | vs Group1 | | |
| Δ_{D 21-D 0} | Mean (sd) of | −0.17 | 1.71 |
| | absolute difference | (1.67) | (1.91) |
| | Timepoints comparison | p = 1 | p = |
| | vs D 0 | | 0.019 |
| | Mean relative | 0% | 4% |
| | difference | | |
| Day 42 | Mean (sd) | 43.31 | 42.04 |
| (D 42) | | (3.42) | (3.68) |
| | Group comparison | | p = |
| | vs Group1 | | 0.978 |
| Δ_{D 42-D 0} | Mean (sd) of | −0.27 | 0.42 |
| | absolute difference | (2.7) | (2.56) |
| | Timepoints comparison | p = 1 | p = 1 |
| | vs D 0 | | |
| | Mean relative | 0% | 1% |
| | difference | | |
| Day 63 | Mean (sd) | 44.19 | 43.2 |
| (D 63) | | (3.37) | (3.32) |
| | Group comparison | | p = |
| | vs Group1 | | 0.998 |
| Δ_{D 63-D 0} | Mean (sd) of | 0.62 | 1.61 |
| | absolute difference | (2.28) | (1.85) |
| | Timepoints comparison | p = | p = |
| | vs D 0 | 0.986 | 0.065 |
| | Mean relative | 2% | 4% |
| | difference | | |

*p is a measure of statistical significance.

The similar or better effects achieved by Example Composition A in comparison to Comparative Composition 1 was unexpected in view of Example Composition A being in the form of a topical serum and Comparative Composition 1 being in the form of a mesotherapy injection composition.

### Cutometric Assessment

Each of the volunteers underwent cutometric assessment at the cheekbone and cheek hollow according to a procedure similar to that described in Example 2.

Table 14 presents a summary of the improvement on the skin firmness, measured at the cheekbone, provided by Example Composition A and Comparative Composition 1.

TABLE 14

| | | Comparative | Example |
|---|---|---|---|
| Firmness or Maximum Amplitude | | Composition | Composition |
| (Cheekbone Skin) | | 1 | A |
| Initial | Mean (sd) | 0.262 | 0.26 |
| Evaluation | | (0.075) | (0.071) |
| (D 0) | Group comparison | | p = 1 |
| | vs Group1 | | |
| Day 21 | Mean (sd) | 0.271 | 0.279 |
| (D 21) | | (0.067) | (0.088) |
| | Group comparison | | p = 1 |
| | vs Group1 | | |
| Δ_{D 21-D 0} | Mean (sd) of | 0.008 | 0.018 |
| | absolute difference | (0.093) | (0.072) |
| | Timepoints comparison | p = 1 | p = |
| | vs D 0 | | 0.996 |
| | Mean relative | 9% | 9% |
| | difference | | |
| Day 42 | Mean (sd) | 0.304 | 0.293 |
| (D 42) | | (0.082) | (0.07) |
| | Group comparison | | p = 1 |
| | vs Group1 | | |
| Δ_{D 42-D 0} | Mean (sd) of | 0.043 | 0.032 |
| | absolute difference | (0.108) | (0.091) |
| | Timepoints comparison | p = | p = |
| | vs D 0 | 0.392 | 0.715 |
| | Mean relative | 23% | 17% |
| | difference | | |
| Day 63 | Mean (sd) | 0.333 | 0.305 |
| (D 63) | | (0.114) | (0.091) |
| | Group comparison | | p = |
| | vs Group1 | | 0.984 |
| Δ_{D 63-D 0} | Mean (sd) of | 0.071 | 0.045 |
| | absolute difference | (0.087) | (0.074) |
| | Timepoints comparison | p = | p = |
| | vs D 0 | 0.004 | 0.314 |
| | Mean relative | 29% | 19% |
| | difference | | |

*p is a measure of statistical significance.

A summary of the improvement on the extend of recovery of the skin length, measured at the cheekbone, provided by Example Composition A and Comparative Composition 1 is presented in Table 15.

TABLE 15

| Extent of Recovery of the Skin | | Comparative | Example |
|---|---|---|---|
| Length (Cheekbone Skin) | | Composition 1 | Composition A |
| Initial | Mean (sd) | 0.579 | 0.565 |
| Evaluation | | (0.097) | (0.086) |
| (D 0) | Group comparison | | p = 1 |
| | vs Group1 | | |
| Day 21 | Mean (sd) | 0.598 | 0.594 |
| (D 21) | | (0.074) | (0.085) |
| | Group comparison | | p = 1 |
| | vs Group1 | | |
| Δ_{D 21-D 0} | Mean (sd) of | 0.019 | 0.029 |
| | absolute difference | (0.099) | (0.098) |
| | Timepoints comparison | p = 1 | p = |
| | vs D 0 | | 0.994 |
| | Mean relative | 5% | 7% |
| | difference | | |
| Day 42 | Mean (sd) | 0.595 | 0.595 |
| (D 42) | | (0.113) | (0.086) |
| | Group comparison | | p = 1 |
| | vs Group1 | | |
| Δ_{D 42-D 0} | Mean (sd) of | 0.017 | 0.032 |
| | absolute difference | (0.112) | (0.104) |
| | Timepoints comparison | p = 1 | p = |
| | vs D 0 | | 0.989 |
| | Mean relative | 4% | 8% |
| | difference | | |
| Day 63 | Mean (sd) | 0.629 | 0.585 |
| (D 63) | | (0.15) | (0.091) |
| | Group comparison | | p = 0.967 |
| | vs Group1 | | |
| Δ_{D 63-D 0} | Mean (sd) of | 0.051 | 0.025 |
| | absolute difference | (0.145) | (0.088) |
| | Timepoints comparison | p = | p = |
| | vs D 0 | 0.74 | 0.999 |
| | Mean relative | 10% | 6% |
| | difference | | |

*p is a measure of statistical significance.

Table 16 presents a summary of the improvement of the skin's net elasticity, measured at the cheekbone, provided by Example Composition A and Comparative Composition 1.

TABLE 16

| | | Comparative | Example |
|---|---|---|---|
| | | Composition | Composition |
| Net Elasticity (Cheekbone Skin) | | 1 | A |
| Initial | Mean (sd) | 0.555 | 0.543 |
| Evaluation | | (0.113) | (0.107) |
| (D 0) | Group comparison | | p = 1 |
| | vs Group1 | | |
| Day 21 | Mean (sd) | 0.557 | 0.558 |
| (D 21) | | (0.083) | (0.093) |
| | Group comparison | | p = 1 |
| | vs Group1 | | |
| Δ_{D 21-D 0} | Mean (sd) of | 0.002 | 0.016 |
| | absolute difference | (0.112) | (0.115) |
| | Timepoints comparison | p = 1 | p = 1 |
| | vs D 0 | | |
| | Mean relative | 4% | 6% |
| | difference | | |
| Day 42 | Mean (sd) | 0.537 | 0.537 |
| (D 42) | | (0.116) | (0.1) |
| | Group comparison | | p = 1 |
| | vs Group1 | | |
| Δ_{D 42-D 0} | Mean (sd) of | −0.017 | −0.003 |
| | absolute difference | (0.107) | (0.112) |
| | Timepoints comparison | p = 1 | p = 1 |
| | vs D 0 | | |
| | Mean relative | −1% | 2% |
| | difference | | |
| Day 63 | Mean (sd) | 0.584 | 0.545 |
| (D 63) | | (0.165) | (0.119) |
| | Group comparison | | p = 0.997 |
| | vs Group1 | | |
| Δ_{D 63-D 0} | Mean (sd) of | 0.03 | 0.008 |
| | absolute difference | (0.125) | (0.116) |
| | Timepoints comparison | p = | p = 1 |
| | vs D 0 | 0.997 | |
| | Mean relative | 6% | 3% |
| | difference | | |

*p is a measure of statistical significance.

A summary of the improvement of the skin fatigue effect, measured at the cheekbone, provided by Example Composition A and Comparative Composition 1 is presented in Table 17.

TABLE 17

| | | Comparative | Example |
|---|---|---|---|
| Skin fatigue effect | | Composition | Composition |
| (Cheekbone Skin) | | 1 | A |
| Initial | Mean (sd) | 0.029 | 0.028 |
| Evaluation | | (0.008) | (0.007) |
| (D 0) | Group comparison | | p = 1 |
| | vs Group 1 | | |
| Day 21 | Mean (sd) | 0.032 | 0.034 |
| (D 21) | | (0.005) | (0.007) |
| | Group comparison | | p = |
| | vs Group 1 | | 0.999 |
| Δ_{D 21-D 0} | Mean (sd) of | 0.003 | 0.006 |
| | absolute difference | (0.009) | (0.01) |
| | Timepoints comparison | p = | p = |
| | vs D 0 | 0.983 | 0.145 |
| | Mean relative | 16% | 32% |
| | difference | | |
| Day 42 | Mean (sd) | 0.032 | 0.031 |
| (D 42) | | (0.008) | (0.007) |
| | Group comparison | | p = 1 |
| | vs Group 1 | | |
| Δ_{D 42-D 0} | Mean (sd) of | 0.003 | 0.003 |
| | absolute difference | (0.009) | (0.01) |
| | Timepoints comparison | p = | p = |
| | vs D 0 | 0.931 | 0.961 |
| | Mean relative | 14% | 20% |
| | difference | | |
| Day 63 | Mean (sd) | 0.036 | 0.032 |
| (D 63) | | (0.006) | (0.009) |
| | Group comparison | | p = |
| | vs Group 1 | | 0.97 |
| Δ_{D 63-D 0} | Mean (sd) of | 0.006 | 0.005 |
| | absolute difference | (0.009) | (0.011) |
| | Timepoints comparison | p = | p = |
| | vs D 0 | 0.087 | 0.579 |
| | Mean relative | 27% | 24% |
| | difference | | |

*p is a measure of statistical significance.

As noted above, the similar or better effects achieved by Example Composition A in comparison to Comparative Composition 1 were surprising.

### Example 5

A non-limiting, example skin care composition (Example Composition B) was prepared in the form of a serum in accordance with aspects of the invention. The formulation of Example Composition B is shown in Table 22 (below).

TABLE 22

| | Ex. B |
|---|---|
| INCI Compound Name | (wt. %) |
| Water | 61-70 |
| Methylpropanediol | 10 |
| Isopentyldiol | 10 |
| Glycerin | 5-5.4 |
| Polysorbate 80 | 3.8-4.4 |
| 1,2-Hexanediol | 1.9-2.2 |
| Fragrance | 0.5 |
| Niacinamide | 0.3-0.5 |
| Propanediol | 0.3-0.5 |
| Taurine | 0.2-0.3 |
| Polyglyceryl-10 Dioleate | 0.2-0.3 |
| Sodium PCA | 0.2-0.3 |
| Carnosine | 0.2-0.3 |
| Mannitol | 0.2-0.3 |
| Sodium Hyaluronate | 0.1-0.3 |
| Tocopheryl Acetate | 0.1-0.3 |
| Citric Acid | 0.1-0.3 |
| Pentylene Glycol | 0.1-0.2 |
| Arginine | 0.1-0.2 |
| Caprylic/Capric Triglyceride | 0.1-0.2 |
| O-CYMEN-5-OL | 0.01-0.1 |
| Hydrolyzed Hyaluronic Acid | 0.01-0.1 |
| Adenosine | 0.01-0.1 |
| Polyglyceryl-10 Dipalmitate | 0.01-0.1 |
| Chondrus Crispus Powder | 0.01-0.1 |
| Silybum Marianum Extract | 0.01-0.1 |
| Sodium Benzoate | 0.01-0.1 |
| Xanthan Gum | 0.01-0.1 |
| Glyceryl Caprylate | 0.01-0.05 |
| Dipeptide Diaminobutyroyl Benzylamide Diacetate | 0.01-0.05 |
| Tocopherol | 0.001-0.01 |
| Benzoic Acid | 0.001-0.01 |
| Glycine | 0.001-0.01 |
| Pseudoalteromonas Ferment Extract | 0.001-0.01 |
| Nicotinamide Adenine Dinucleotide | 0.001-0.01 |
| Magnesium Sulfate | 0.001-0.01 |
| Ergothioneine | 0.001-0.01 |
| Colorants | 0.0002 |

### Example 6

Example Compositions A and B were evaluated in comparison to three comparative compositions (Comparative Compositions 2-4) to assess the efficacy of the respective compositions. A list of ingredients for Comparative Compositions 2 is provided below.
Water, Glycereth-26, Glycerin, Methylpropanediol, Pentylene Glycol, Sodium Chloride, Ppg-26-Buteth-26, Peg-40 Hydrogenated Castor Oil, Phenoxyethanol, Parfum (Fragrance), Sodium Hyaluronate, Propanediol, Sucrose Palmitate, Glucose, Xanthan Gum, Tocopheryl Acetate, Disodium Edta, Octadecyl Di-T-Butyl-4-Hydroxyhydrocinnamate, Potassium Chloride, Glyceryl Linoleate, Prunus Amygdalus Dulcis (Sweet Almond) Oil, Calcium Chloride, Adenosine, Magnesium Sulfate, Glutamine, Sodium Hydroxide, Sodium Phosphate, Ascorbic Acid, Sodium Acetate, Glyceryl Caprylate, Lysine Hcl, Arginine Hcl, Alanine, Histidine Hcl, Valine,Leucine, Threonine, Citric Acid, Isoleucine, Tryptophan, Phenylalanine, Tyrosine, Glycine, Polysorbate 80, Potassium Sorbate, Serine, Cystine, Cyanocobalamin, Glutathione, Asparagine, Aspartic Acid, Pseudoalteromonas Ferment Extract, Ornithine Hcl, Glutamic Acid, Nicotinamide Adenine Dinucleotide, Proline, Methionine, Taurine, Hydroxyproline, Glucosamine Hcl, Coenzyme A, Tocopherol, Sodium Glucuronate, Thiamine Diphosphate, Retinyl Acetate, Inositol, Niacin, Niacinamide, Pyridoxine Hcl, Biotin, Calcium Pantothenate, Riboflavin, Sodium Tocopheryl Phosphate, Thiamine Hcl, and Folic Acid.

A list of ingredients for Comparative Compositions 3 is provided below.
Water\Aqua\Eau, Bifida Ferment Lysate, Peg-8, Propanediol, Bis-Peg-18 Methyl Ether Dimethyl Silane, Methyl Gluceth-20, Glycereth-26, Peg-75, Butylene Glycol, Adansonia Digitata Seed Extract, Tripeptide-32, Sodium Hyaluronate, Yeast Extract\Faex\Extrait De Levure, Lactobacillus Ferment, Cola Acuminata (Kola) Seed Extract, Anthemis Nobilis (Chamomile) Flower Extract, Hydrolyzed Algin, Pantethine, Caffeine, Lecithin, Sodium Rna, Bisabolol, Squalane, Glycerin, Oleth-3 Phosphate, Caprylyl Glycol, Oleth-3, Oleth-5, Choleth-24, Hydrogenated Lecithin, Jojoba Wax Peg-120 Esters, Ceteth-24, Tocopheryl Acetate, Carbomer, Triethanolamine, Tetrasodium Edta, Bht, Xanthan Gum, Potassium Sorbate, Disodium Edta, Phenoxyethanol, Red 4 (Ci 14700), and Yellow 5 (Ci 19140).

A list of ingredients for Comparative Compositions 4 is provided below.
Water, Bifida Ferment Lysate, Glycerin, Alcohol Denat., Dimethicone, Hydroxyethylpiperazine Ethane Sulfonic Acid, Ascorbyl Glucoside, Lactobacillus, Tocopherol, Sodium Hyaluronate, Sodium Hydroxide, Sodium Benzoate, Phenoxyethanol, Adenosine, Faex Extract/Yeast Extract, Peg-20 Methyl Glucose Sesquistearate, Peg-60 Hydrogenated Castor Oil, Polymnia Sonchifolia Root Juice, Maltodextrin, Salicyloyl Phytosphingosine, Ammonium Polyacryloyldimethyl Taurate, Limonene, Mannose, Xanthan Gum, Pentylene Glycol, Propylene Glycol, Caprylyl Glycol, Alpha-Glucan Oligosaccharide, Sorbitol, Disodium Edta, Rosa Gallica Flower Extract, Octyldodecanol, Citronellol, and Fragrance.

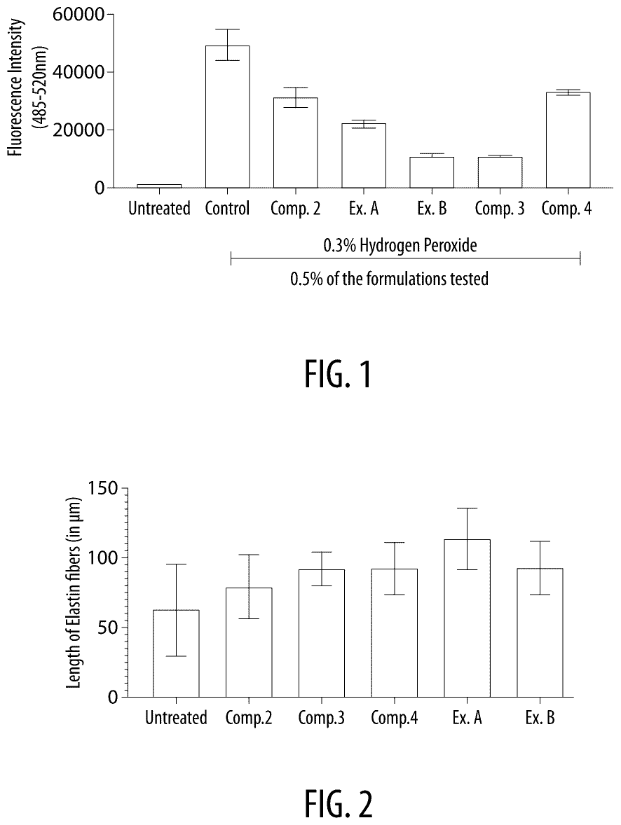

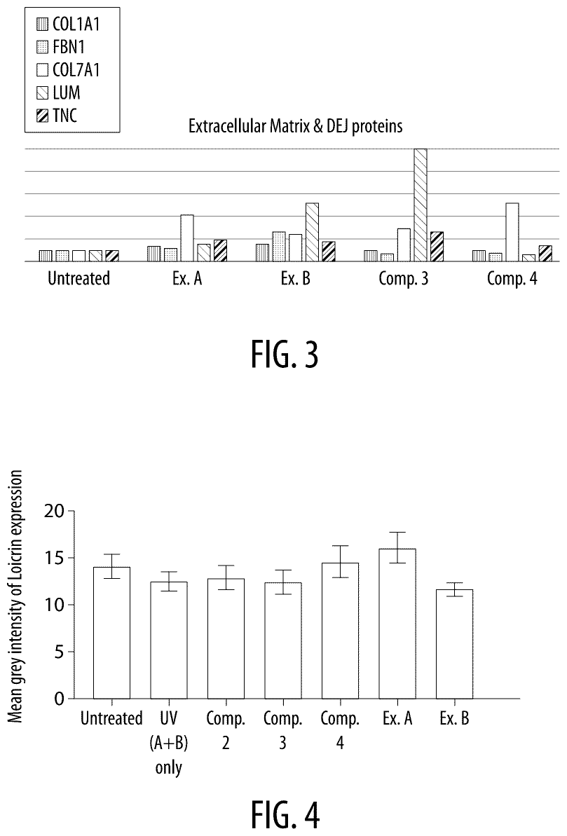

Specifically, the reduction of oxidative species (ROS), elasticity, (ECM), Barrier, and Vitality were evaluated for each of Example Compositions A and B as well as Comparative Compositions 2-4. A summary of the evaluation of Example Compositions A and B in comparison to Comparative Compositions 2-4 is shown in FIGS. 1-4. Specifically, FIG. 1 is a bar graph of the effect on the reduction of oxidative species. FIG. 2 is a bar graph of the effect on elastin fibers. FIG. 3 is a bar graph of the effect on COL1A1, FBN1, COL7A1, LUM, and TNC expression. FIG. 4 is a bar graph of the effect on loricrin expression.

### Example 7

Example Composition A was clinically evaluated by performing an in vivo clinical assessment and a self-evaluation assessment. Thirty Caucasian female volunteers of ages from 35 to 65 years with skin presenting redness and discomfort sensation were selected for the evaluation of Example Composition A.

The volunteers underwent a washout period of about two weeks before applying Example Composition A. After the washout period, Example Composition A was applied twice a day, specifically once in the morning and once in the evening, for 28 days.

The volunteers were evaluated visually under standard “daylight.” Standardized photographic images were obtained with normal, cross polarized and UV lamp for both hemifaces and the front using a system VISIA-CR system in order to quantify the evolution of the effect. The measuring area was the face (both the full face and profile). A VISIA-CR imaging system is a standardized imaging photographic system that takes high resolution photos with a Standard Light IntelliFlash, a Cross Polarized flash, and under an Ultraviolet lighting. The analysis software allows a definition of a Region of Interest and the calculation of the areas and number of the parameters. The measurements were performed after first application (“D0”), after 7 days of application (“D7”), and also at the end of the study after 28 days of application (“D28”). The redness parameter was calculated based on the full aligned image.

Concurrently with the clinical examinations, each volunteer was questioned about possible sensations of discomfort they felt. The volunteers were requested to note every day any reaction observed and any possible sensation of discomfort felt on the individual observation sheet they were given at the beginning of the study.

Regarding the red spots count, Example Composition A presented a 2.8%, 10.1% and 9.8% decrease after the first application, after 7 days of application, and after 28 days of application, respectively. The reduction in the red spots count was statistically significant. Regarding the red spots' area, Example Composition A presented a 7.9%, 18.8% and 20.1% decrease after the first application, after 7 days of application, and after 28 days of application, respectively. The reduction in the red spots' area was also statistically significant. Additionally, Example Composition A presented a 3.9%, 4.4% and 22.8% improvement in clinical erythema assessment score, after the first application, after 7 days of application, and after 28 days of application, respectively. The improvement in clinical erythema assessment score was statistically significant.

Regarding the self-assessment scores by the subjects, Example Composition A presented a 38.4%, 40.7% and 81.3% improvement in discomfort sensation score, after the first application, after 7 days of application, and after 28 days of application, respectively. The improvement in discomfort sensation score was statistically significant.

### Example 8

Example Composition A from Example 1 was evaluated in comparison to four comparative compositions (Comparative Compositions 5-8). The formulations for Example Composition A and Comparative Compositions 5-8 are shown in Table 23. Other than the amounts of water, propanediol, glycerin, pentylene glycol, 1,2-hexanediol, xanthan gum, fragrances, colorants, and preservatives, the amounts for each of the ingredients when present in the respective formulation were the same for Example Composition A and Comparative Compositions 5-8.

TABLE 23

| | Ex. A | Comp. 5 | Comp. 6 | Comp. 7 | Comp. 8 |
|---|---|---|---|---|---|
| INCI Compound Name | (wt. %) | (wt. %) | (wt. %) | (wt. %) | (wt. %) |
| Water | 78-84 | 88.4 | 85 | 85 | 85 |
| Propanediol | 5.1-5.5 | 5 | 5.4 | 5.4 | 5.4 |
| Glycerin | 5-5.4 | 0.1 | 2.9 | 2.9 | 2.9 |
| Pentylene Glycol and/or 1,2-Hexanediol | 1.1-1.3 | 1 | 1 | 1 | 1 |
| Caprylic/Capric Triglyceride | 0.1-0.3 | | | | |
| Polyglyceryl-10 Dioleate | 0.1-0.3 | | | | |
| PEG-40 Hydrogenated Castor Oil | 1.9-2.2 | 1.9-2.2 | 1.9-2.2 | 1.9-2.2 | 1.9-2.2 |
| Triethyl Citrate | 1.9-2.2 | 1.9-2.2 | 1.9-2.2 | 1.9-2.2 | 1.9-2.2 |
| Xanthan Gum | 0.05-0.1 | 0.05 | 0.06 | 0.06 | 0.06 |
| Tocopherol | 0.001-0.01 | | | | |
| Tocopheryl Acetate | 0.1-0.3 | | | | |
| Sodium PCA | 0.1-0.3 | | | 0.1-0.3 | |
| Ergothioneine | 0.001-0.005 | | | 0.001-0.005 | |
| Niacinamide | 0.4-0.6 | | | 0.4-0.6 | |
| Carnosine | 0.1-0.3 | | | | |
| Sodium Hyaluronate | 0.1-0.3 | | 0.1-0.3 | 0.1-0.3 | 0.1-0.3 |
| Hydrolyzed Hyaluronic Acid | 0.01-0.1 | | | 0.01-0.1 | |
| Arginine | 0.1-0.3 | | | 0.1-0.3 | |
| Adenosine | 0.01-0.05 | 0.01-0.05 | 0.01-0.05 | 0.01-0.05 | 0.01-0.05 |
| Polyglyceryl-10 Dipalmitate | 0.03-0.05 | | | | |
| Silybum Marianum Extract | 0.01-0.04 | | | | 0.01-0.04 |
| Glyceryl Caprylate | 0.01-0.05 | | 0.01-0.05 | 0.01 | 0.01 |
| Dipeptide Diaminobutyroyl Benzylamide Diacetate | 0.01-0.03 | | 0.01-0.03 | 0.01-0.03 | 0.01-0.03 |
| Mannitol | 0.1-0.3 | | | 0.1-0.3 | |
| Glycine | 0.001-0.005 | | | 0.001-0.005 | |
| Pseudoalteromonas Ferment Extract | 0.001-0.003 | | 0.001-0.003 | 0.001-0.003 | 0.001-0.003 |
| Magnesium Sulfate | 0.001-0.003 | | | 0.001-0.003 | |
| Nicotinamide Adenine Dinucleotide | 0.001-0.003 | | | 0.001-0.003 | |
| Taurine | 0.1-0.4 | | | 0.1-0.4 | |
| Colorants | 0.0001-0.001 | 0.0001-0.001 | 0.0001-0.001 | 0.0001-0.001 | 0.0001-0.001 |
| Fragrance | 0.3 | 0.3 | 0.3 | 0.3 | 0.3 |
| Preservative (Citric Acid, Sodium Citrate, | 1.2 | 1.1 | 1.1 | 1.1 | 1.1 |
| Benzoic Acid, Sodium Benzoate, and | | | | | |
| Benzotriazolyl Dodecyl P-Cresol) | | | | | |

Example Composition A was evaluated to assess its effect on senescence biomarkers relative to Comparative Compositions 5-8. Specifically, 3D full thickness skin models (EFT400 from MatTek) were treated topically for 6 days in culture. Samples of Example Composition A and Comparative Compositions 5-8 were applied to the skin samples and then incubated at a temperature of 37°° C. in an environment having 5 vol. % of carbon dioxide. A control sample was prepared evaluating a skin sample that was not treated with one of Example Composition A or Comparative Compositions 5-8, but otherwise underwent the same experimental procedures. Following incubation, the epidermis from the skin samples was collected, the RNA therefrom was isolated, and the expression of actively transcribed genes was analyzed with qPCR. The results were quantified and graphed as fold expression relative to the untreated control, which is set to 1. Fold expression values of greater than 1 indicate upregulation/stimulation of gene expression, while expression values of less than 1 indicate downregulation/inhibition of expression.

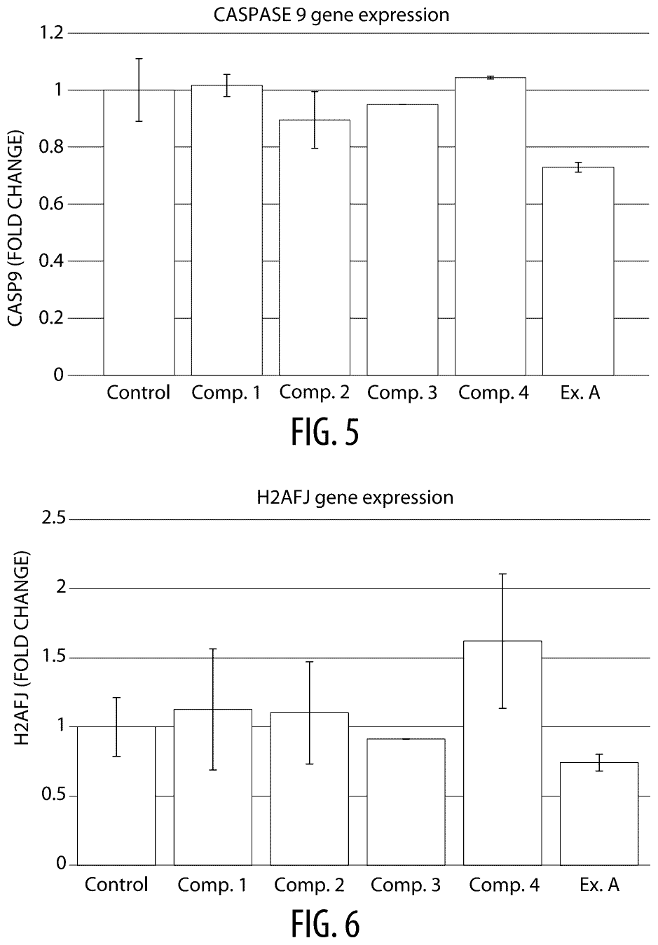

A summary of the evaluation of the expression of actively transcribed genes is shown in FIGS. 5 and 6. Specifically, FIG. 5 is a bar graph of the CASPASE 9 gene expression while FIG. 6 is a bar graph of the H2AFJ gene expression.

What is claimed is:

1. A skin care composition comprising:
a hyaluronic acid or a salt thereof;
a vitamin E;
a peptide;
an extract comprising one or more of silybum marianum extract or pseudoalteromonas ferment extract; and
water in an amount of about 45 wt. % or more, based on the total weight of the skin care composition.

2. The skin care composition according to claim 1, wherein the hyaluronic acid or a salt thereof comprises hydrolyzed hyaluronic acid.

3. The skin care composition according to claim 1, wherein the hyaluronic acid or a salt thereof comprises sodium hyaluronate.

4. The skin care composition according to claim 1, wherein the vitamin E is selected from alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, and a combination of two or more thereof.

5. The skin care composition according to claim 1, wherein the peptide comprises dipeptide diaminobutyroyl benzylamide diacetate, acetyl tetrapeptide-11, acetyl tetrapeptide-9, palmitoyl tripeptide-1, palmitoyl tetrapeptide-7, or a combination of two or more thereof.

6. The skin care composition according to claim 1, wherein the extract comprises both silybum marianum extract and pseudoalteromonas ferment extract.

7. The skin care composition according to claim 1 further comprising:
one or more polyol in an amount from about 4 to about 22 wt. %, based on the total weight of the skin care composition, wherein the one or more polyol comprises glycerin, ethylene glycol, propylene glycol, butylene glycol, caprylyl glycol hexylene glycol, diethylene glycol, dipropylene glycol, or a combination of two or more thereof.

8. The skin care composition according to claim 1 further comprising:
a nonionic surfactant in an amount from about 0.1 to about 5 wt. %, based on the total weight of the skin care composition, wherein the nonionic surfactant is selected from pegylated castor oils, polyglycerol esters, poloxamers, fatty alcohol ethoxylates, alkylphenol ethoxylates, tertiary amine oxides, glucosides, and a combination of two or more thereof.

9. The skin care composition according to claim 8, wherein the pegylated castor oil has a pegylated chain of about 10 to about 80.

10. The skin care composition according to claim 1 further comprising:
an amino acid component comprising ergothioneine and at least one amino acid selected from the group consisting of taurine, arginine, glycine, and a combination of two or more thereof.

11. The skin care composition according to claim 10, wherein the amino acid component comprises ergothioneine, taurine, arginine, and glycine.

12. The skin care composition according to claim 1 further comprising:
a vitamin B present in an amount from about 0.1 to about 3 wt. %, based on the total weight of the skin care composition, wherein the vitamin B comprises niacinamide.

13. The skin care composition according to claim 1 further comprising:
a pyrrolidone carboxylic acid and/or a salt thereof present in an amount from about 0.1 to about 3 wt. %, based on the total weight of the skin care composition.

14. The skin care composition according to claim 13, wherein the skin care composition has a weight ratio of pyrrolidone carboxylic acid and/or a salt thereof to niacinamide of from about 1:1 to about 1:11.

15. The skin care composition according to claim 11, wherein the skin care composition has a weight ratio of alpha-tocopherol to ergothioneine of about 6:1 to about 1:6.

16. The skin care composition according to claim 11, wherein the skin care composition has a weight ratio of alpha-tocopherol to niacinamide of from about 1:1 to about 1:11.

17. The skin care composition according to claim 11, wherein the skin care composition has a weight ratio of ergothioneine to niacinamide of from about 1:1 to about 1:11.

18. The skin care composition according to claim 1, wherein the water is present in an amount from about 60 to about 90 wt. %, based on the total weight of the skin care composition.

19. A method of reducing wrinkles and/or improving skin elasticity, homogeneity, luminosity, and/or firmness, the method comprising:
injecting a composition to an area of skin; and
applying a serum to the area of skin, the serum comprising:
hyaluronic acid or a salt thereof,
vitamin E,
a peptide,
an extract comprising one or more of silybum marianum extract or pseudoalteromonas ferment extract, and
water in an amount of about 45 wt. % or more, based on the total weight of the skin care composition.

20. The method according to claim 19, wherein the serum is applied to the area of skin within 72 hours of the injection of the composition.
